(19) United States
(12) Patent Application Publication
(10) Pub. No.: US 2023/0174611 A1
(43) Pub. Date: Jun. 8, 2023

(54) COMPOSITIONS AND METHODS FOR PREVENTING OR REDUCING THE EFFECTS OF INFECTIONS BY CORONAVIRUSES THAT BIND THE EXTRACELLULAR DOMAIN OF THE ACE2 RECEPTOR

(71) Applicant: Administrators of the Tulane Educational Fund, New Orleans, LA (US)

(72) Inventors: Jay K KOLLS, New Orleans, LA (US); Naoki IWANAGA, New Orleans, LA (US)

(73) Assignee: Administrators of the Tulane Educational Fund, New Orleans, LA (US)

(21) Appl. No.: 17/919,507

(22) PCT Filed: Apr. 26, 2021
(86) PCT No.: PCT/US2021/029121

§ 371 Date: Oct. 17, 2022

Related U.S. Application Data
(60) Provisional application No. 63/014,777, filed on Apr. 24, 2020.
(60) Provisional application No. 63/114,325, filed on Nov. 16, 2020.

(51) Int. Cl. C07K 14/705; A61P 31/14

(52) U.S. Cl. CPC C07K 14/705; A61P 31/14; C07K 2319/30

(57) ABSTRACT

The invention provides compositions and methods for preventing, or reducing the effects of, an infection by coronaviruses, including SARS-CoV-2, that bind human ACE2. The compositions are fusion proteins with mutated forms of the extracellular domain of the ACE2 receptor which can bind viral particles of these coronaviruses. When sprayed or inhaled into an individual’s nasal passages or airway, the inventive fusion proteins bind particles of such coronaviruses, keeping them from reaching and infecting the individual’s cells. When administered parenterally, the fusion proteins can enter the fluid lining the inside of the lung, binding particles of such coronaviruses and keeping them from binding to and infecting cells.

### CROSS-REFERENCE TO RELATED APPLICATIONS

This application claims priority to, and the benefit of, U.S. Provisional Pat. Application No. 63/114,325, filed Nov. 16, 2020, and U.S. Provisional Pat. Application No. 63/014,777, filed Apr. 24, 2020. The contents of both applications are incorporated herein by reference for all purposes.

### STATEMENT OF FEDERAL FUNDING

This invention was made with government support under Grant R35HL139930 awarded by the National Heart, Lung, and Blood Institute of the National Institutes of Health. The government has certain rights in the invention.

### BACKGROUND OF THE INVENTION

Middle East respiratory syndrome coronavirus (MERS-CoV), severe acute respiratory syndrome coronavirus (SARS-CoV), and severe acute respiratory syndrome coronavirus 2 (SARS-CoV-2) are the causative agents of Middle East respiratory syndrome (MERS), severe acute respiratory syndrome (SARS), and severe acute respiratory syndrome 2, respectively. Severe acute respiratory syndrome 2 can, in turn, cause coronavirus disease 2019 (COVID-19). The current outbreak of SARS-CoV-2 that was identified in December 2019 was declared a pandemic by the World Health Organization in March 2020 and as of Apr. 22, 2020, had resulted in over 2,400,000 confirmed cases and over 169,000 confirmed deaths worldwide, even as the outbreak was still increasing. SARS-CoV and SARS-CoV-2 cause respiratory disease by infecting cells of the respiratory tract through the receptor angiotensin converting enzyme 2 (ACE2).

Compounds that can inhibit viral entry to cells are potential therapeutic targets for such coronaviruses. In particular, soluble ACE2 has been shown to inhibit viral entry of SARS-CoV. However, the half-life of soluble ACE2 is likely too short to be therapeutic in vivo. Additionally, soluble ACE2 has no active transport mechanism from the circulation into the alveolar spaces of the lung, a critical site of infection.

It would be useful to have agents that can inhibit entry of coronaviruses or other viruses that enter cells through the ACE2 receptor, thereby reducing the severity of infection by such viruses, or preventing infection by such viruses. It would further be useful to have agents that can be transported into the lung or nasal passages when administered by intravenous or subcutaneous routes. Surprisingly, the present invention fills these and other needs.

### BRIEF SUMMARY OF THE INVENTION

The present invention provides compositions, uses, products and and methods for reducing the risk of infection by coronaviruses that enter cells by binding the ACE2 receptor or for slowing viral progression and reducing symptoms in persons who have been infected by such coronaviruses, including SARS-CoV-2.

In a first group of embodiments, the invention provides compositions comprising a fusion protein of formula 1,

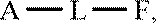

wherein
“A” is a modified extracellular domain of a human ACE2 receptor, which extracellular domain has a wild-type sequence has an N-terminal amino acid residue and an ability to catalyze angiotensin, which extracellular domain has been mutated to reduce or to eliminate said ability of said extracellular domain to catalyze angiotensin,
“L” is a short peptide linker, and
“F” is the fragment crystallizable region (“Fc” or “Fc region”) of an antibody, optionally wherein the Fc region has been mutated to eliminate FcRy binding, but to retain binding to a neonatal Fc receptor. In some embodiments, the N-terminal amino acid residue is a serine, glutamine, aspartic acid, glutamic acid, threonine, or asparagine residue. In some embodiments, the N-terminal amino acid residue is a serine, a glutamine, or an aspartic acid residue. In some embodiments, the mutated human ACE2 extracellular domain has a non-polar, uncharged amino acid residue at position 345 with reference to SEQ ID NO:1. In some embodiments, the non-polar, uncharged amino acid residue at position 345 is valine, isoleucine, leucine, glycine, or alanine. In some embodiments, the non-polar, uncharged amino acid residue at position 345 is an alanine. In some embodiments, the mutated human ACE2 extracellular domain has a non-polar, uncharged amino acid residue at position 273 with reference to SEQ ID NO:1. In some embodiments, the non-polar, uncharged amino acid residue at position 273 is valine, isoleucine, leucine, glycine, or alanine. In some embodiments, the non-polar, uncharged amino acid residue at position 273 is an alanine. In some embodiments, the mutated human ACE2 extracellular domain has a non-polar, uncharged amino acid residue at both position 273 and at position 345, which non-polar, uncharged amino acid residue at each position may be the same or different. In some embodiments, the non-polar, uncharged amino acid residue at position 273 and at position 345 is an alanine. In some embodiments, the short peptide linker is from 3 to 20 amino acids in length. In some embodiments, the short peptide linker is from 3 to 12 amino acids in length. In some embodiments, the short peptide linker is from 3 to 8 amino acids in length. In some embodiments, the short peptide linker is I-E-G-R (SEQ ID NO:2). In some embodiments, the short peptide linker is L-V-P-R-G-S (SEQ ID NO:4), A-A-A-A (SEQ ID NO:5), or A-A-A-A-A (SEQ ID NO:6), or has the formula GGGGS₍ₙ₎, wherein n=5 or fewer, preferably four or fewer. In some embodiments, the short peptide linker is G-G-G-G-S (SEQ ID NO:15). In some embodiments, the short peptide linker is G-G-G-G-S-G-G-G-G-S (SEQID NO:16). In some embodiments, the short peptide linker is G-G-G-G-S-G-G-G-G-S-G-G-G-G-S (SEQ ID NO:16). In some embodiments, the short peptide linker is G-G-G-G-S-G-G-G-G-S-G-G-G-G-S-G-G-G-G-S (SEQ ID NO:17). In some embodiments, the Fc is of IgG1, mutated to have alanines in place of leucines at the positions corresponding to positions 18 and 19 of SEQ ID NO:3 (an exemplar Fc sequence is shown in FIG. 10, with the positions mutated to alanines shown in bold face and underlined). In some embodiments, the fusion protein is MDR504-Fc (SEQ ID NO:11). In some embodiments, the fusion proteins is MDR504-WL-Fc (SEQ ID NO:19), MDR504S-WL-Fc (SEQ ID NO:21) or MDR504Q-WL-Fc (SEQ ID NO:23). In some embodiments, the fusion protein is MDR505-Fc (SEQ ID NO: 12). In some embodiments, the fusion protein is MDR505-WL-Fc (SEQ ID NO:20), MDR505S-WL-Fc (SEQ ID NO:22) or MDR505Q-WL-Fc (SEQ ID NO:24). In some embodiments, the Fc region has been mutated to contain LALA mutations, a YTE mutation, or both LALA mutations and a YTE mutation. In some embodiments, the composition further comprises a pharmaceutically acceptable carrier.

In a second group of embodiments, the invention provides for the use of the compositions set forth above for the manufacture of a medicament for the prevention of, or the reduction of severity of, an infection by a coronavirus that binds to human ACE2 receptor. In some embodiments, the coronavirus is SARS-CoV-2. In some embodiments, the mutated human ACE2 extracellular domain of said composition has a non-polar, uncharged amino acid residue at position 345, as that position is numbered in SEQ ID NO:1. In some embodiments, the non-polar, uncharged amino acid residue at position 345 is valine, isoleucine, leucine, glycine, or alanine. In some embodiments, the non-polar, uncharged amino acid residue at position 345 is an alanine. In some embodiments, the mutated human ACE2 extracellular domain of said composition has a non-polar, uncharged amino acid residue at position 273, as that position is numbered in SEQ ID NO:1. In some embodiments, the non-polar, uncharged amino acid residue at position 273 is an alanine. In some embodiments, the mutated human ACE2 extracellular domain has a non-polar, uncharged amino acid residue at both position 273 and at position 345, which non-polar, uncharged amino acid residue at each position is the same or different. In some embodiments, the non-polar, uncharged amino acid residue at position 273 and at position 345 is an alanine. In some embodiments, the composition of claim 1 is MDR504-Fc (SEQ ID NO:11). In some embodiments, the composition of claim 1 is MDR504-WL-Fc (SEQ ID NO:19), MDR504S-WL-Fc (SEQ ID NO:21), or MDR504Q-WL-Fc (SEQ ID NO:23). In some embodiments, the composition of claim 1 is MDR505-Fc (SEQ ID NO:12). In some embodiments, the composition of claim 1 is MDR505-WL-Fc (SEQ ID NO:20), MDR505S-WL-Fc (SEQ ID NO:22), or MDR505Q-WL-Fc (SEQ ID NO:24). In some embodiments, the Fc region has been mutated to contain LALA mutations, a YTE mutation, or both LALA mutations and a YTE mutation. In some embodiments, the composition is suitable for aerosol or nasal administration. In some embodiments, the composition is suitable for intravenous administration.

In a third group of embodiments, the invention provides products comprising a composition of claim 1 for the prevention of, or the reduction of severity of, an infection by a coronavirus that binds to human ACE2 receptor. In some embodiments, the coronavirus is SARS-CoV-2. In some embodiments, the composition of claim 1 is MDR504-Fc (SEQ ID NO: 11). In some embodiments, the composition of claim 1 is MDR504-WL-Fc (SEQ ID NO:19), MDR504S-WL-Fc (SEQ ID NO:21), or MDR504Q-WL-Fc (SEQ ID NO:23). In some embodiments, the composition of claim 1 is MDR505-Fc (SEQ ID NO:12). In some embodiments, the composition of claim 1 is MDR505-WL-Fc (SEQ ID NO:20), MDR505S-WL-Fc (SEQ ID NO:22), or MDR505Q-WL-Fc (SEQ ID NO:24). In some embodiments, the composition of claim 1 has LALA mutations, a YTE mutation, or both LALA mutations and a YTE mutation. In some embodiments, the composition is in a pharmaceutically acceptable carrier, is mixed with a pharmaceutically acceptable excipient, or both.

In yet another group of embodiments, the invention provides methods of preventing or of ameliorating an infection in a subject by a coronavirus that binds to a human ACE2 receptor, said method comprising administering to said subject an effective amount of a pharmaceutical composition comprising a fusion protein of formula 1,

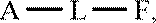

“A” is a mutated extracellular domain of a human ACE2 receptor, which extracellular domain has a wild-type sequence has an N-terminal amino acid residue and an ability to catalyze angiotensin, which extracellular domain has been mutated to reduce or to eliminate said ability of said extracellular domain to catalyze angiotensin,
“L” is a short peptide linker, and
“F” is the fragment crystallizable region (“Fc” or “Fc region”) of an antibody, optionally wherein said Fc region has been mutated to eliminate FcRy binding, but to retain binding to a neonatal Fc receptor. In some embodiments, the N-terminal amino acid residue of the “A” component residue is a serine, glutamine, aspartic acid, glutamic acid, threonine, or asparagine residue. In some embodiments, the N-terminal amino acid residue is a glutamine residue, serine residue or an aspartic acid residue. In some embodiments, the mutated human ACE2 extracellular domain has a non-polar, uncharged amino acid residue at a position corresponding to position 345 of SEQ ID NO:1. In some embodiments, the non-polar, uncharged amino acid residue at position 345 is valine, isoleucine, leucine, glycine, or alanine. In some embodiments, the non-polar, uncharged amino acid residue at the position corresponding to position 345 of SEQ ID NO:1 is an alanine. In some embodiments, the mutated human ACE2 extracellular domain has a non-polar, uncharged amino acid residue at a position corresponding to position 273 of SEQ ID NO:1. In some embodiments, the non-polar, uncharged amino acid residue at the position corresponding to position 273 of SEQ ID NO:1 is valine, isoleucine, leucine, glycine, or alanine. In some embodiments, the non-polar, uncharged amino acid residue at the position corresponding to position 273 of SEQ ID NO:1 is an alanine. In some embodiments, the mutated human ACE2 extracellular domain has a non-polar, uncharged amino acid residue at both the position corresponding to position 273 of SEQ ID NO:1 and at the position corresponding to position 345 of SEQ ID NO:1, which non-polar, uncharged amino acid residue at each position is the same or different. In some embodiments, the non-polar, uncharged amino acid residue at said position corresponding to position 273 of SEQ ID NO:1 and at the position corresponding to position 345 of SEQ ID NO:1 is an alanine. In some embodiments, the short peptide linker is from 3 to 20 amino acids in length. In some embodiments, the short peptide linker is from 3 to 12 amino acids in length. In some embodiments, the short peptide linker is from 3 to 8 amino acids in length. In some embodiments, the short peptide linker is I-E-G-R (SEQ ID NO:2). In some embodiments, the short peptide linker is L-V-P-R-G-S (SEQ ID NO:4), A-A-A-A (SEQ ID NO:5), A-A-A-A-A (SEQ ID NO:6), or has the formula GGGGS₍ₙ₎. In some embodiments, the short peptide linker is G-G-G-G-S (SEQ ID NO:15). In some embodiments, the Fc is of IgG1, mutated to have alanines in place of leucines at positions 234 and 235 (SEQ ID NO:3). In some embodiments, the fusion protein is MDR504-Fc (SEQ ID NO:11). In some embodiments, the fusion proteins is MDR504-WL-Fc (SEQ ID NO:19), MDR504S-WL-Fc (SEQ ID NO:21) or MDR504Q-WL-Fc (SEQ ID NO:23). In some embodiments, the fusion protein is MDR505-Fc (SEQ ID NO:12). In some embodiments, the fusion protein is MDR505-WL-Fc (SEQ ID NO:20), MDR505S-WL-Fc (SEQ ID NO:22) or MDR505Q-WL-Fc (SEQ ID NO:24). In some embodiments, the Fc of said composition of claim 1 has LALA mutations, a YTE mutation, or both LALA mutations and a YTE mutation. In some embodiments, the composition further comprises a pharmaceutically acceptable carrier, a pharmaceutically acceptable excipient, or both.

### BRIEF DESCRIPTION OF THE DRAWINGS

FIG. 1. FIG. 1 is a schematic diagram of an embodiment of the inventive fusion proteins, comprising a mutated human sACE2 ectodomain, a peptide linker, and the Fc region of a human immunoglobulin. In the embodiment depicted, the Fc linker is mutated from the natural sequence of the human Fc region of the immunoglobulin from which it is derived.

FIGS. 2A-2E. FIGS. 2A-2E are graphs show the results of studies of in vitro binding to either the spike protein of SARS-CoV-2 (“spike protein”) or the receptor binding domain (“RBD”) of the spike protein by fusion proteins made with either the native human ACE2 ectodomain linked by a peptide linker to a mutated Fc region (“wt hACE2-Fc”), or one of three fusion proteins, MDR503hACE2-FC, MDR504 hACE2-Fc, of MDR505 hACE2-Fc, each of which has the same peptide linker and Fc region as wt hACE2Fc, but with a different mutated human ACE2 ectodomain. For each of these Figures, the Y axis shows the absorption at 450 nm (“OD450”), while the X axis shows the concentration in µM. FIG. 2A. FIG. 2A shows ELISA data from the binding of the four fusion proteins to RBD at room temperature. FIG. 2B. FIG. 2B shows ELISA data from the binding of the four fusion proteins to spike protein at room temperature. FIG. 2C. FIG. 2C shows ELISA data from the binding of the four fusion proteins to the spike protein RBD at 37° C. FIG. 2D. FIG. 2D shows ELISA data from the binding of the four fusion proteins to spike protein at 37° C.

FIGS. 3A and 3B. FIG. 3A. FIG. 3A is a graph showing SARS-CoV2 viral neutralization in vitro with WT hACE2-Fc, and mutants MDR503, MDR504, and MDR505, analyzed by plaque assay. Y axis: % number of plaques. X axis: log concentration (nM). FIG. 3B. FIG. 3B is a table presenting the calculated IC50 in nm (mean ± s.d.) of each construct in the plaque assay.

FIGS. 4A and 4B. FIG. 4A. FIG. 4A is a graph showing in vivo pharmacokinetics of wild-type ACE2-Fc and the MDR504 ACE2-Fc mutant fusion proteins assayed at the time of intravenous injection of 4 mg/kg body weight of the designated fusion protein in serum and at 1 hour, 24 hours, and 72 hours after injection. FIG. 4B. FIG. 4B is a graph showing in vivo pharmacokinetics of wild type ACE2-Fc and the MDR504 ACE2-Fc mutant fusion proteins assayed at the time of intravenous injection of 4 mg/kg body weight of the designated fusion protein in bronchoalveolar lavage fluid (“BALF”) and at 6 hours and 72 hours after injection. For each graph, n=3.

FIGS. 5A-5E. In vivo prophylaxis using an exemplar fusion protein in an animal model. FIGS. 5A-5E present results of in vivo prophylaxis in a mild-to-moderate COVID-2 infection in a mouse model. Mice were induced to express hACE2 via delivery of adeno 5-drived hACE2 viruses to their lung (Ad5-hACE2) were infected with SARS-CoV-2. Three to four hours prior to infection, mice were left untreated or were dosed intravenously with 15 mg/kg with one of human polyclonal IgG1, WT hACE2-Fc, or MDR504-Fc. FIG. 5A. FIG. 5A presents micrographs of immunohistochemistry of mice that were given oropharyngeal inoculation of Ad5-hACE2, four hours before SARS-CoV-2 challenge, treated intravenously with 15 mg/kg body weight human polyclonal IgG1, WT hACE2-Fc, or MDR504 hACE2-Fc, and then sacrificed and studied three days later. White: DAPI stain. Green: SARS-CoV-2. FIG. 5B. FIG. 5B is a graph of data from the mice discussed with respect to FIG. 5A, showing, on the Y axis, the CoV2 cell area in % and, on the X axis, the agent (human polyclonal IgG1, WT hACE2-Fc, or MDR504 hACE2-Fc ) with which the mice had been treated prior to infection with SARS-CoV-2. FIG. 5C. FIG. 5C presents photomicrographs of RNA Scope performed on tissue from the mice discussed in FIG. 5A. Magnification is 100x. (Red: SARS-CoV-2 S, Blue: Cxcl9). FIG. 5D. FIG. 5D presents a graph of Cxcl9 gene expression in animals that have not been infected, animals that were infected but not treated, and animals that were infected after prophylactic treatment with either WT hACE2-Fc, or MDR504 hACE2-Fc. Gene expression was assessed by RT-PCR. Significant differences are designated using one-way ANOVA followed by Tukey’s multiple comparisons test. *, P < 0.05, **, P < 0.01, ***, P < 0.001, ****, P < 0.0001. FIG. 5E. FIG. 5E presents a graph of Cxcl0 gene expression in animals that have not been infected, animals that were infected but not treated, and animals that were infected after prophylactic treatment treated with either WT hACE2-Fc, or MDR504 hACE2-Fc. Gene expression was assessed by RT-PCR. Significant differences are designated using one-way ANOVA followed by Tukey’s multiple comparisons test. *, P < 0.05, **, P < 0.01, ***, P < 0.001, ****, P < 0.0001.

FIGS. 6A-F. In vivo treatment using an exemplar fusion protein in a severe COVID-19 mouse model. K18-hACE2 humanized mice, which support higher levels of SARS-CoV-2 replication than Ad5-ACE2 mice, were infected with SARS-CoV-2 and left untreated or treated with either WT hACE2-Fc, or the exemplar fusion protein MDR504 hACE2-Fc four hours after infection. FIG. 6A. FIG. 6A is a graph showing the viral load in animals left untreated or treated with either WT hACE2-Fc, or with MDR504 hACE2-Fc, as measured by total N gene expression. FIG. 6B. FIG. 6B is a graph showing the viral load in animals left untreated or treated with either WT hACE2-Fc, or with MDR504 hACE2-Fc, as measured by subgenomic N gene expression. FIG. 6C. FIG. 6C is a graph of Cxcl9 gene expression in animals that were infected and left untreated, and in animals that were infected and treated with either WT hACE2-Fc, or with MDR504 hACE2-Fc. Gene expression was measured by RT-PCR. Significant differences are designated using Kruskal-Wallis test followed by Dunn’s multiple comparisons test. *, P < 0.05, **, P < 0.01 FIG. 6D. FIG. 6D is a graph of weight loss in mice with human ACE2 receptor that were infected with SARS-CoV-2 and either left untreated (administered just vehicle, n=12) or treated with either WT hACE2-Fc (n=7), or with MDR504 hACE2-Fc (n=8). Y axis: weight loss (ratio). X axis: days after challenge.

$$† \text{p < 0} \text{.05}$$

and

$$† † \text{p < 0} \text{.01}$$

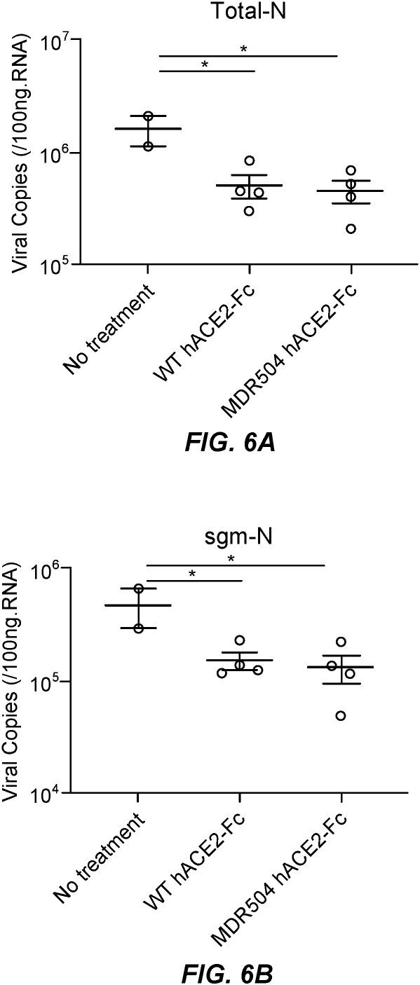

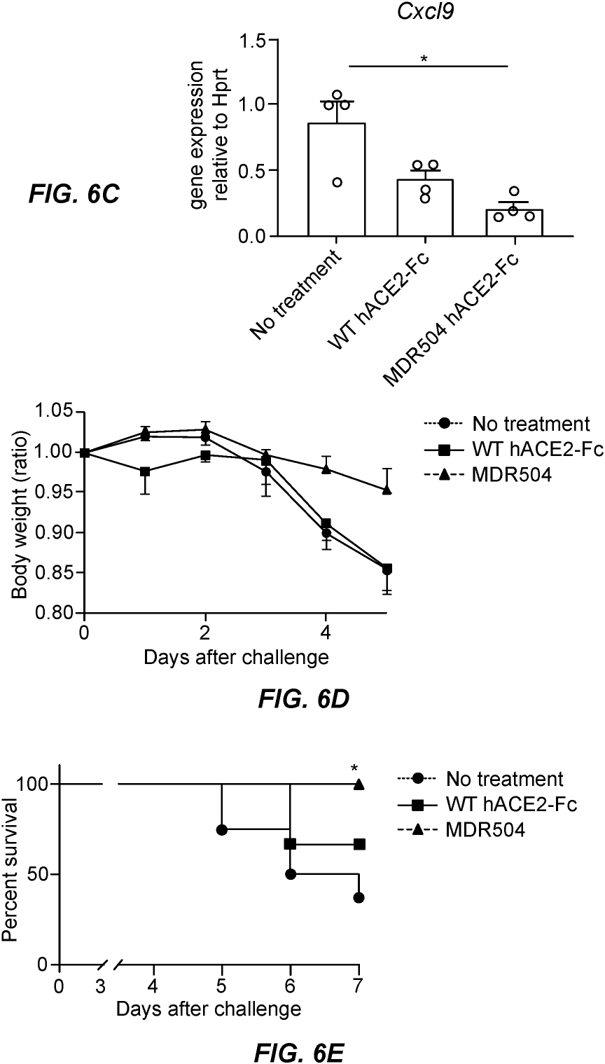

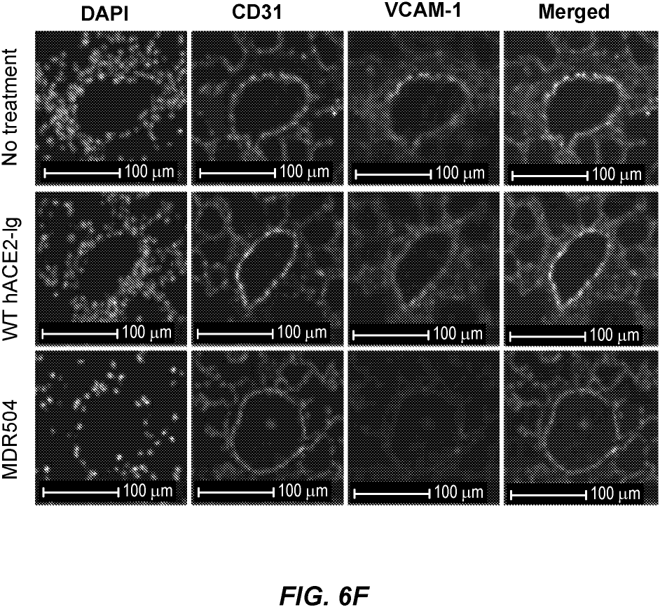

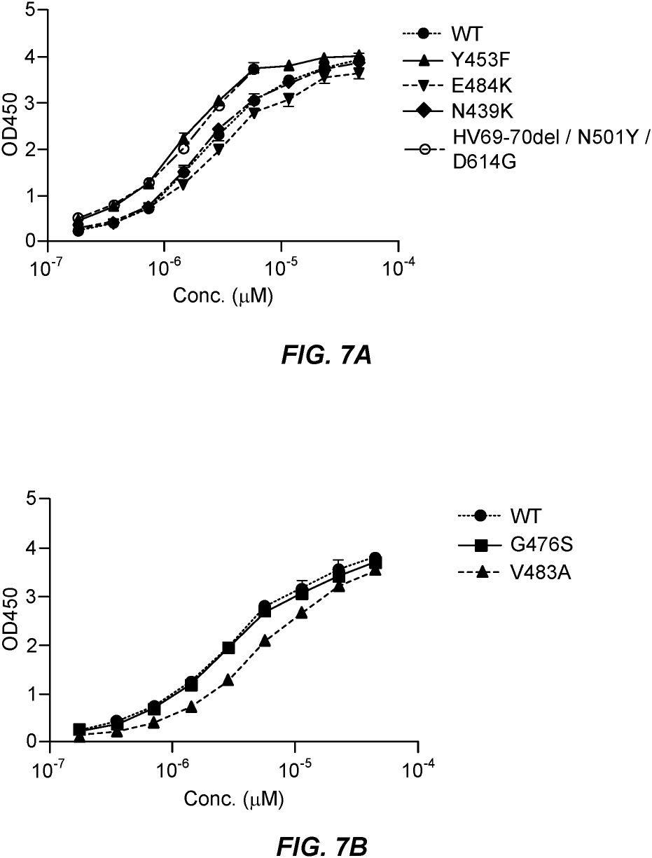

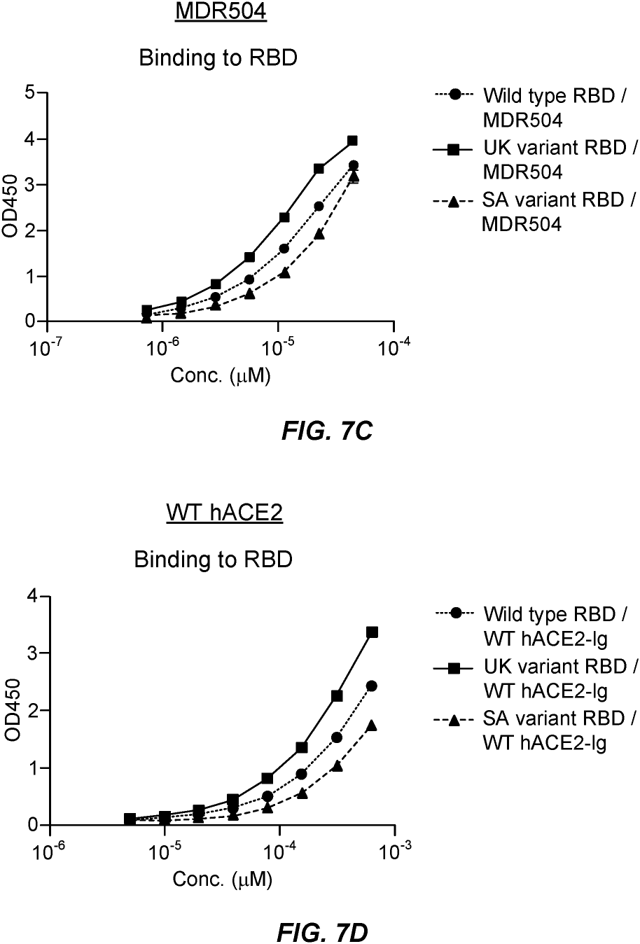

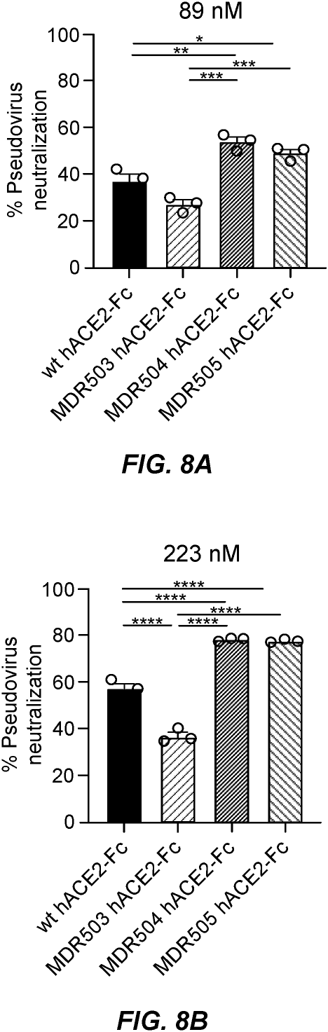

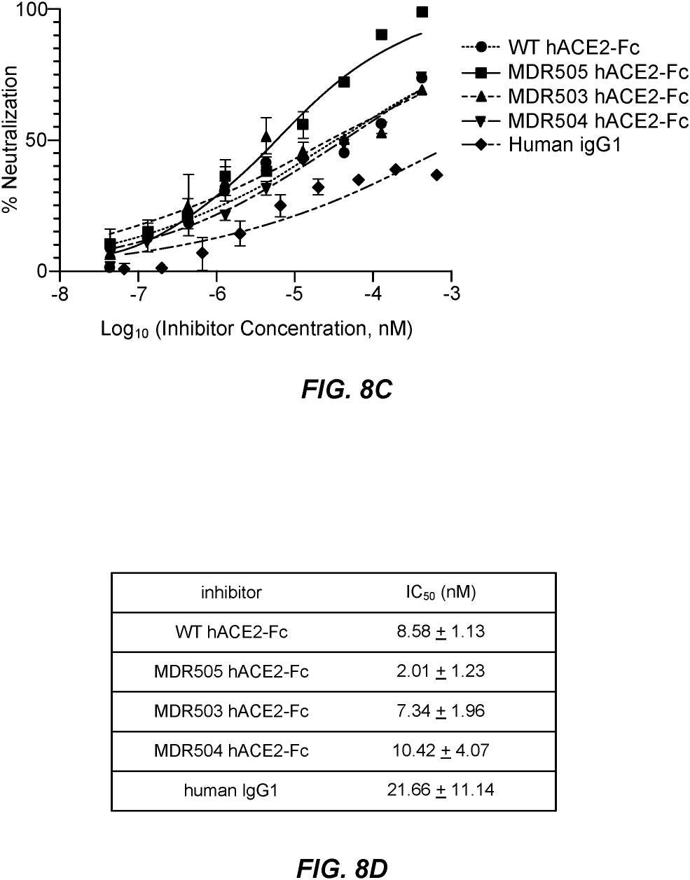

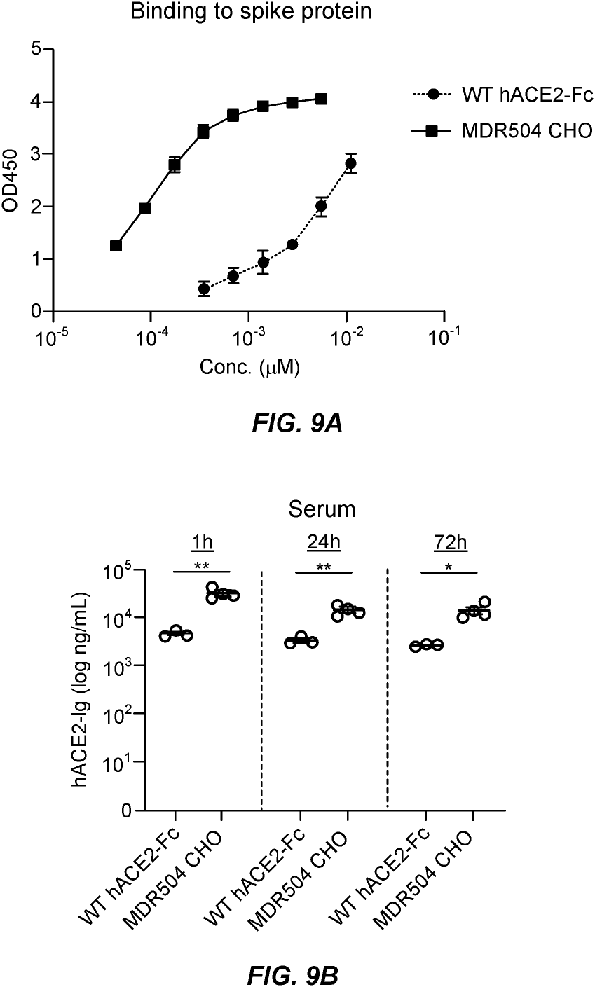

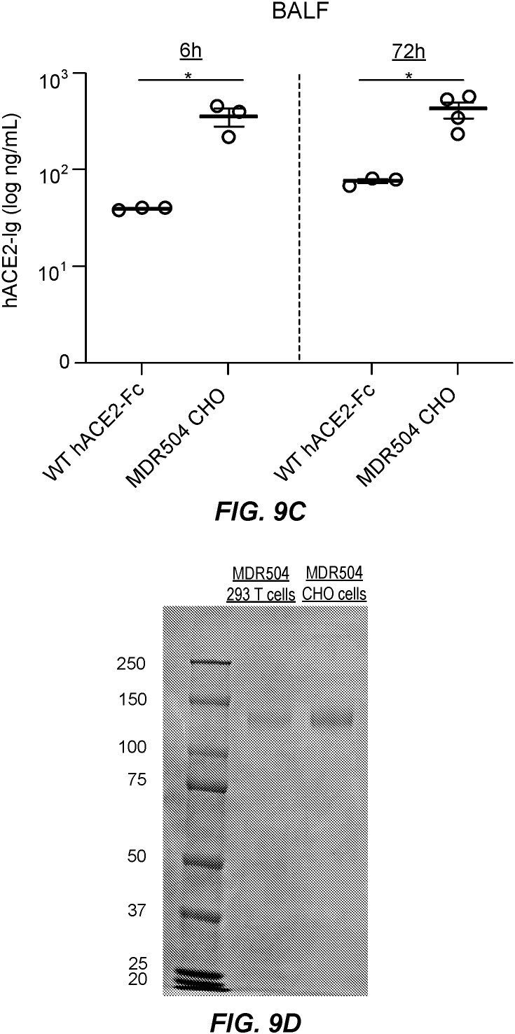

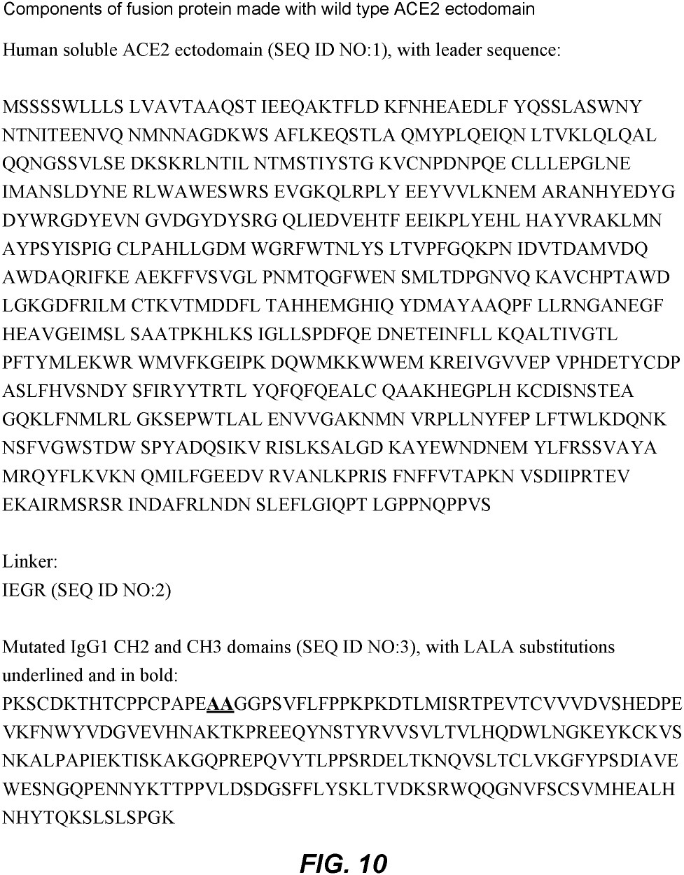

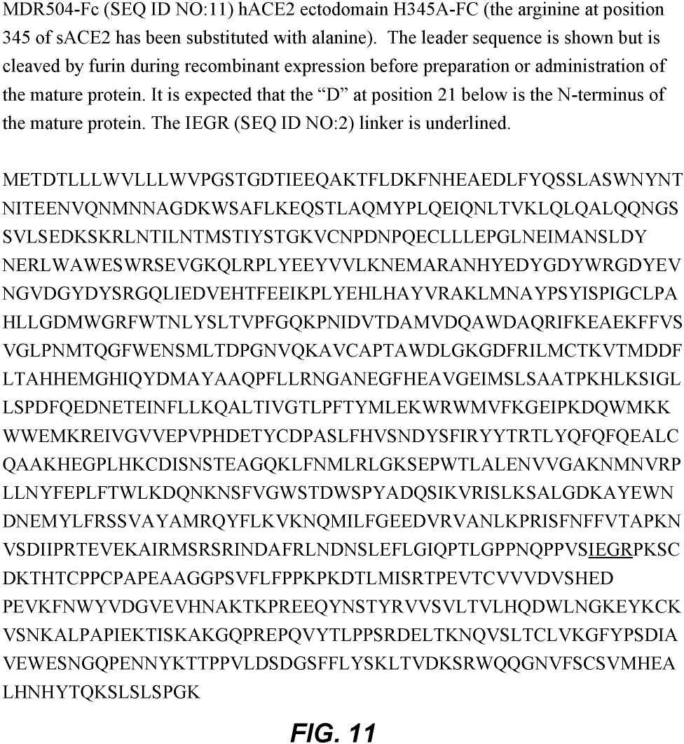

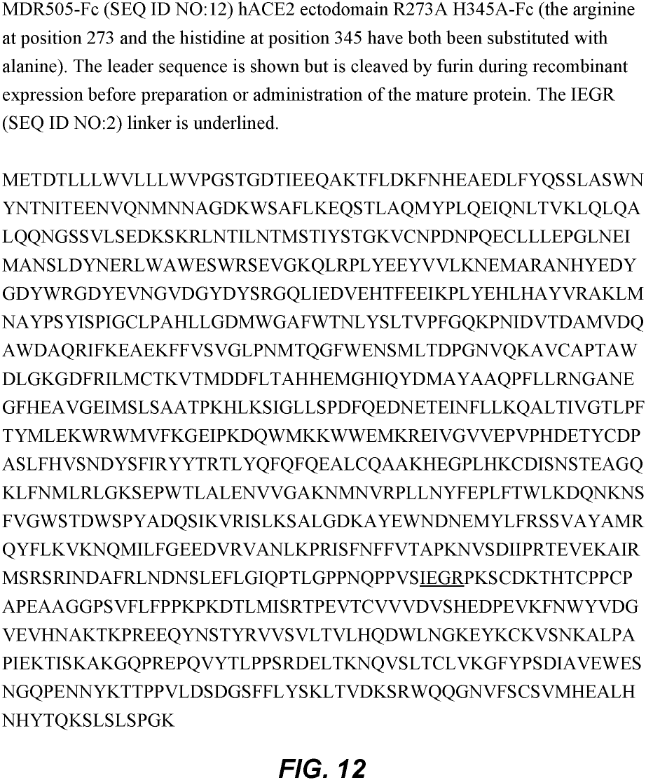

(vs WT hACE2-Fc), ***p < 0.001 and ****p < 0.0001 (vs No treatment) by 2-way ANOVA. FIG. 6E. FIG. 6E is a graph of the percent of mice with human ACE2 receptor that were infected with SARS-CoV-2 surviving after infection when either given no treatment (administered just vehicle, n=12), or treated with either WT hACE2-Fc (n=7), or with MDR504 hACE2-Fc (n=8). Y axis: percent survival. X axis: days after challenge. P < 0.05 log-rank test. FIG. 6F. FIG. 6F sets forth photographs at 40x (top row) or 100x (bottom row) showing staining for VCAM-1 in cells from lungs of animals with human ACE2 receptor that were infected with SARS-CoV-2 and then given only vehicle (“no treatment”), or treatment with either WT hACE2-Fc, or with MDR504 hACE2-Fc.

FIGS. 7A-7D. FIG. 7A. FIG. 7A is a graph showing the binding of exemplar fusion protein MDR504 hACE2-Fc to SARS-CoV-2 spike protein with either the sequence considered to be the wild-type sequence (“WT”) or mutations of potential concern. The graph states the particular mutations of the spike protein against which binding of the fusion protein was tested. Y axis: optical density measured at 450 nm. X axis: concentration of MDR504 hACE2-Fc in µM. FIG. 7B. FIG. 7B is a graph showing the binding of exemplar fusion protein MDR504 hACE2-Fc to SARS-CoV-2 spike protein with either the sequence considered to be the wild-type sequence (“WT”) or with selected mutations of potential concern. The graph states the particular mutations of the spike protein against which binding of the fusion protein was tested. Y axis: optical density measured at 450 nm. X axis: concentration of MDR504 hACE2-Fc in µM. FIG. 7C. FIG. 7C is a graph showing the binding of exemplar fusion protein MDR504 hACE2-Fc to the SARS-CoV-2 spike protein receptor binding domain (“RBD”) with either the sequence considered to be the wild-type sequence (“WT”) or the sequence of the U.K or of the South African SARS-CoV-2 variants. Y axis: optical density measured at 450 nm. X axis: concentration of MDR504 hACE2-Fc in µM. FIG. 7D. FIG. 7D is a graph showing the binding of the WT hACE2 fusion protein to the same SARS-CoV-2 spike protein RBDs as described for FIG. 7C. Y axis: optical density measured at 450 nm. X axis: concentration of MDR504 hACE2-Fc in µM

FIGS. 8A-8D. Comparison of the ability of different constructs to neutralize pseudovirus in vitro. FIG. 8A. FIG. 8A is a graph showing the ability of the different fusion proteins tested to neutralize pseudovirus, The concentration used for each fusion protein listed was 89 nm. FIG. 8B. FIG. 8B is a graph showing the ability of the different fusion proteins tested to neutralize pseudovirus, The concentration used for each fusion protein was 223 nm. For both FIGS. 8A and 8B, significant differences are designated using one-way ANOVA followed by Tukey’s multiple comparisons test. *, P < 0.05; **, P < 0.01; ***, P < 0.001; ****, P < 0.0001. (n = 3). FIG. 8C. FIG. 8C is a graph of a dose response curve. The MDR505 hACE2-Fc mutant showed particularly higher neutralization. The error bar represents the s.d. of three replicates. All data was normalized to virus alone. FIG. 8D. FIG. 8D is a table showing the calculated IC₅₀ (mean ± s.d.) of each construct based on the SARS-CoV-2 pseudovirus neutralization.

FIGS. 9A-9D. FIG. 9A. The fusion proteins used in the studies reported in this disclosure were produced in 293T cells and in CHO cells. FIG. 9A. FIG. 9A is a graph showing that an exemplar fusion protein of the invention, MDR504-Fc, produced in CHO cells (“MDR504 CHO”) showed much higher binding to SARS-CoV-2 spike protein than did a fusion protein with the same linker peptide and Fc region, but with the wild-type hACE2 ectodomain (“WT hACE2-Fc”) instead of the mutated ectodomain used in the MDR504 fusion protein. FIG. 9B. FIG. 9B is a graph comparing in vivo pharmacokinetics of wild-type hACE2-Fc and the MDR504 ACE2-Fc mutant fusion proteins produced in CHO cells, by intravenously injecting 4 mg/kg body weight of the designated fusion protein and assaying the serum at 1 hour, 24 hours, and 72 hours after injection. FIG. 9C. FIG. 9C is a graph comparing in vivo pharmacokinetics of wild-type hACE2-Fc and the MDR504 ACE2-Fc mutant fusion proteins produced in CHO cells, by intravenously injecting 4 mg/kg body weight of the designated fusion protein and assaying the fusion proteins present in bronchoalveolar lavage BAL fluid (“BALF”) at 6 hours and 72 hours after injection. For each graph, n=3. FIG. 9D. FIG. 9D is a photograph of a western blot showing that the molecular weight of the MDR504-Fc fusion protein produced in CHO cells (“MDR504 CHO cells”) was nearly identical to that of the same fusion protein produced in 293T cells (“MDR504 293T cells”). Any actual difference in molecular weight is believed to be due to differences in glycosylation patterns of the fusion protein depending on the cells in which it is expressed.

FIG. 10. FIG. 10 sets out the amino acid sequence of a fusion protein which, for clarity is separated into (a) the wild-type hACE2 ectodomain (SEQ ID NO:1) sequence, including the leader directing the expressed protein into the secretory pathway and cleaved during secretion, (b) an exemplar short peptide linker, IEGR (SEQ ID NO:2), and an exemplar IgG1 Fc region, with LALA mutations (SEQ ID NO:3). The two alanines introduced into the Fc region by the LALA mutations are underlined. Though shown separately in the Figure, the components are expressed as a single fusion protein.

FIG. 11. FIG. 11 sets out the amino acid sequence of a sACE2-Fc fusion protein in which the soluble hACE2 ectodomain sequence (SEQ ID NO:1), with the leader cleaved off before or during secretion from host cells expressing the fusion protein, has been mutated to contain a mutation of H to A at position 345 (“H345A”). The hACE2 ectodomain is linked through an exemplar peptide linker, IEGR (SEQ ID NO:2) (underlined) to an exemplar IgG1 Fc with LALA mutations (SEQ ID NO:3). The resulting fusion protein (SEQ ID NO:11) is referred to herein as “MDR504-Fc” or “MDR504 construct” (SEQ ID NO:11), with the presence of the linker being understood.

FIG. 12. FIG. 9 sets out the amino acid sequence of a sACE2-Fc fusion protein in which the soluble hACE2 ectodomain sequence (SEQ ID NO:1) has been mutated to contain both (i) a mutation of arginine (R) to alanine (A) at position 273, and (ii) a mutation of histidine (H) to alanine (A) at position 345. The hACE2 ectodomain is linked through an exemplar peptide linker, IEGR (SEQ ID NO:2) (underlined) to an exemplar IgG1 Fc with LALA mutations (SEQ ID NO:3). The resulting fusion protein (SEQ ID NO:12) is usually referred to herein as “MDR505-Fc” or “MDR505 construct,” with the presence of the linker being understood.

### DETAILED DESCRIPTION

SARS-CoV-2 is the etiologic agent of the current COVID-19 pandemic. Similar to SARS-CoV and other betacoronaviruses, SARS-CoV-2 uses angiotensin-converting enzyme 2 (“ACE2”) to enter cells (see, e.g., Xu et al., Int J Oral Sci. 2020;12(1):8. Epub 2020/02/26. doi: 10.1038/s41368-020-0074-x). ACE2 is a transmembrane protein that has an extracellular domain, a transmembrane domain, and an intracellular domain. The extracellular domain of the ACE2 protein functions both as an enzyme and as a receptor. ACE2 is expressed in the nasal respiratory epithelium, in the conducting airway in type II pneumocytes (Hou et al., Cell. 2020. Epub 2020/06/12. doi: 10.1016/j.cell.2020.05.042), and elsewhere (Hoffman et al., Cell. 2020;181(2):271-80 e8. Epub 2020/03/07. doi:10.1016/j.cell.2020.02.052). It has been hypothesized that the spike protein of SARS-CoV-2 has stronger binding to the human ACE2 receptor than does SARS-CoV. The nucleic acid and translated amino acid sequence of full-length human ACE2, including the leader sequence which is cleaved during secretion of the mature protein, are known and are set forth in GenBank®, at NCBI Reference Sequence: NM_021804.1 The GenBank sequence has been recently updated and the current curated sequence is set forth under NCBI Reference Sequence NM_021804.3. References herein to the sequence of the inventive fusion proteins and to positions of residues within the ACE2 ectodomain are with reference to the sequence of SEQ ID NO:1.

The extracellular domain of ACE2, sometimes referred to as the “ectodomain,” can be cleaved from the other portions of the ACE2 protein, or can be expressed recombinantly without the transmembrane and intracellular portions of ACE2. In these forms, it is known as “soluble ACE2,” which is sometimes abbreviated as “sACE2”. Soluble ACE2 has been shown to bind to SARS-CoV and SARS-CoV-2 spike proteins and block viral entry. (Monteil, et al., Cell. 2020. Epub 2020/04/26. doi: 10.1016/j.cell.2020.04.004).

Soluble ACE2 has been administered to humans with pulmonary hypertension (Hemnes et al., Eur Respir J. 2018;51(6). Epub 2018/06/16. doi: 10.1183/13993003.02638-2017) and acute respiratory distress syndrome (ARDS) (Khan et al., Crit Care. 2017;21(1):234. Epub 2017/09/08. doi: 10.1186/s13054-017-1823-x), at dose ranges of 0.1 to 0.8 mg/kg and shown to be well tolerated. However, the pharmacokinetic/pharmacodynamic (PK/PD) of soluble ACE2 is not ideal for sustained viral neutralization in vivo and it is not transported well from the circulation into the epithelial lining fluid of the lung. Thus, wild-type (“wt”) sACE2 has limited utility for the therapeutic uses noted above and it is not by itself useful as a therapy for, or prophylactic against, SARS-CoV-2 infection. It would be desirable to have forms of sACE2 that have better pharmacokinetics and that can be transported from the circulation into the epithelial lining fluid of the lung. Additionally, SARS-CoV-2 has been found in the kidneys, heart, and other organs of persons with SARS-CoV-2 infections. It would be desirable to have forms of soluble ACE2 that could help reduce infection of organs other than the lung in individuals with SARS-CoV-2 infections.

Surprisingly, we have developed recombinant fusion proteins that solve the problems of pharmacokinetics and transportation of sACE2 to the lung, that have high affinity for the SARS-CoV-2 spike protein, and that, when administered to animals before challenge with SARS-CoV-2, reduce the severity of infection by the virus as measured by immunohistochemistry and by cytokine expression profile. Further, we believe our results indicate that, by extension, our fusion protein constructs will have high affinity to the spike protein of other beta coronaviruses that rely on binding of the ACE2 receptor to enter cells, and will be useful in preventing infection by those viruses as well, and in reducing the symptoms of infection by those viruses, particularly in early stages of infection. References below to “soluble ACE2” refer to the extracellular domain of human ACE2.

In some embodiments, the inventive fusion proteins can be administered by spraying of an aerosol or mist comprising the fusion proteins into the nasal passages, the lung, or both, or by inhalation by the person in need thereof of such an aerosol or mist into the nasal passages, the lung, or both. In some embodiments, the fusion proteins can be administered parenterally to reduce the infection of the lung and of other organs in individuals infected with SARS-CoV-2 and to reduce damage caused by the virus. Without wishing to be bound by theory, it is believed that the fusion proteins bind particles of the SARS-CoV-2 virus, either irreversibly, in which case the virus particles are rendered unable to infect cells of the affected individual, or reversibly, in which case, the binding slows the rate at which free virus particles find and infect cells of the affected individual. This slower progression gives the individual’s immune system more time to develop an immune response, slows the rate at which the patient’s infection progresses, and gives medical providers more time to provide other therapeutic options.

In some embodiments, the inventive fusion proteins can be administered to reduce the chance that SARS-CoV-2 can establish an infection in an individual. For example, if a nursing home is having an outbreak of COVID-19, residents and staff can inhale an aerosolized mist containing the fusion proteins to coat their nasal passages and render it less likely that any SARS-CoV-2 virus particles in the ambient air will find and bind ACE2 receptors on their cells. In some embodiments, the inventive fusion proteins can be administered to the lungs of a person having or suspected of having an infection with SARS-CoV-2 to slow or stop spread of the virus through the individual’s lungs, thereby reducing the chance the virus will exit the individual’s lung and enter the individual’s circulation.

It is expected that one or more of the vaccines against SARS-CoV-2 currently approved for emergency use will be fully approved for use. Even once one or even multiple vaccines become approved, however, there will be individuals who will not be able to be vaccinated for medical reasons, and others who will refuse them due to religious objections or sincere, if uninformed, beliefs about vaccines Such individuals are expected to benefit from prophylactic use of the inventive fusion proteins to reduce their susceptibility to coronaviruses in general or SARS-CoV-2 in particular, or to benefit from therapeutic use of the inventive fusion proteins if they contract COVID-19.

Taken together, the results presented herein show that the inventive fusion proteins are useful for both pre- and post-exposure prophylaxis and are expected to be useful as a therapy for persons with COVID-19, whether caused by the original viral strain or some or all of the variants that have emerged as of this writing. The inventive fusion proteins are particularly likely to be useful for therapeutic use in early stages of SARS-CoV-2 infection.

The inventive fusion proteins are expected to be useful both as a complement to vaccines, and as stand-alone prophylaxis or therapy for subjects that may not be good candidates for vaccines, such as patients with hematologic or other malignancies, those undergoing immunosuppressive therapy for organ transplantation, those with autoimmune disease, and those with HIV or other conditions that prevent them from mounting a robust immune response to a vaccine.

The following section describes the general formula for embodiments of the inventive fusion proteins. The section after that describes exemplar inventive fusion proteins made in the course of the present work.

### The Inventive Fusion Proteins

The inventive fusion proteins can be described generally by Formula 1:

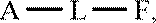

wherein “A” is a sACE2 extracellular domain (SEQ ID NO:1) that has been mutated to to eliminate catalysis of angiotensin by replacing the histidine at the position corresponding to position 345 of SEQ ID NO:1 with a non-polar amino acid, by replacing the arginine at the position corresponding to position 273 of SEQ ID NO:1 with a non-polar amino acid, or by replacing both the specified histidine and the specified arginine with a non-polar amino acid,
“L” is a short peptide linker, and
“F” is the fragment crystallizable region (“Fc” or “Fc region”) of an antibody. In some embodiments, the Fc has been mutated to eliminate FcRy binding (by using a LALA mutation), but to retain binding to the neonatal Fc receptor, which is important for serum stability and transport into the lung. In some embodiments, the Fc has been mutated to increase serum half-life. In some embodiments, the mutation of the Fc is the “YTE” mutation. The YTE mutation and other mutations extending serum half-life are set forth in, e.g., Booth et al., MABS, 2018, 10(7): 1098-1110, https://doi.org/10.1080/19420862.2018.1490119.

As practitioners are aware, proteins like the inventive proteins can be produced by expressing or synthesizing each component separately and then chemically conjugating them, but recombinant technology and expression systems have reached the point where it is faster and easier to express them recombinantly as fusion proteins. While chemically conjugated proteins are expected to be used less commonly than recombinant fusion proteins, they are expected to work similarly, if not identically. For convenience of reference, the term “fusion proteins” as used herein refers both to recombinantly expressed fusion proteins and to chemically conjugated proteins unless otherwise specified or required by context.

For expression as recombinant proteins, the constructs of Formula 1 preferably include a leader sequence that directs the expressed protein to the secretory pathway. The leader sequence is cleaved by endogenous enzymes, such as furin, leaving the mature protein, now lacking the leader sequence, for purification and use. Expression is preferably performed in eukaryotic cells, to allow for proper glycosylation of any glycosylation points on the fusion protein. Leader sequences and the components named in Formula 1 will be discussed in turn in more detail below.

Finally, when amino acids are covalently attached to one another in a peptide or a protein, they are usually referred to as “amino acid residues.” For convenience of reference in describing the components of the inventive fusion proteins, the constituents will be referred to below interchangeably as “amino acids” or “residues,” except when specifying one form or the other is required. For convenience, the amino acids or residues, as appropriate, will usually be referred to herein by the single letter codes adopted by convention to designate individual amino acids.

### Leader Sequences

Fusion proteins intended to be produced in and then secreted by host cells typically are expressed with a leader sequence which directs the leader sequence-fusion protein along the secretory pathway. The leader sequence is then typically cleaved by cellular enzymes before the fusion protein, now free of its leader sequence, is secreted from the host cell, leaving the fusion protein, which may be referred to as the “mature protein,” available for purification and use.

Furin is a cell-encoded proprotein convertase present in the Golgi complex, at the plasma membrane, and within endosomes. The leader sequence used in studies underlying the present disclosure was from IgGκ, but other leader sequences suitable for expression and secretion in mammalian cells are known in the art, as discussed further below. Preferably, the leader sequence used directs proteins of which it is a part to the Golgi apparatus for secretion. Preferably, the leader sequence is cleavable by furin in the Golgi apparatus. Furin is known to preferentially recognize the cleavage site sequence R-X-K/R-R, (See, e.g., Duckert, et al., Protein Eng. Des. Sel., 2004, 17:107-112.pmid: 14985543; Krysan, et al., J. Biol. Chem., 1999, 274:23229-23234.pmid:10438496), and it is accordingly easy to select leader sequences that contain a furin-cleavable sequence at the desired position in the proprotein to result in secretion of the desired fusion protein. It is contemplated that such leader sequences can be substituted for the leader sequences used in the studies reported below. Any particular leader sequence can be readily tested to confirm its suitability by using it as a leader sequence to express one of the fusion proteins discussed in the Examples and determine if the fusion protein is secreted and can be readily detected by ELISA, as discussed in the Examples, below.

Selection of leader sequences suitable for use in a particular expression system using a particular cell type, however, is well known and usually involves criteria such as selecting for leader sequences that do not interfere with high levels of expression of the desired fusion protein and that can be cleaved without cleaving the desired fusion protein. Companies which specialize in expressing recombinant proteins typically have preferred expression systems and preferred leader sequences that work well in those expression systems. For some studies reported below, the fusion proteins were expressed in HEK293T cells or CHO cells using an IgGκ leader sequence. It is expected that the practitioner can select leader sequences suitable for use in other cell types to produce the inventive fusion proteins. Production in eukaryotic cells is preferred.

In some studies underlying the present disclosure, the leader used was that of the human wild-type ACE2 ectodomain sequence (the entire human ACE2 ectodomain sequence is SEQ ID NO:1). A paper by Procko, posted on bioRxiv, May 11, 2020, doi: https://doi.org/1.0.1101/2020.03.16.99423, states that the mature ACE2 protein comprises amino acids 19-805, which implies that the leader is cleaved between residues 18 and 19 of SEQ ID NO:1. The native sequence is MSSSSWLLLSLVAVTAAQ (SEQ ID NO:13) (“Leader sequence 1”). Residue 19 of the wild-type sequence is a serine. Assuming that furin cleaves the leader of the wild-type sequence between residues 18 and 19, the serine residue at position 19 becomes the N-terminus of the mature protein and is part of some embodiments of the inventive fusion proteins. Early studies of human ACE2 indicated that the leader sequence was 17 amino acids (see, e.g., Tipnis et al., J Biol Chem, 2000, 275:33238-43, DOI:https://doi.org/10.1074/jbc.M002615200), in which case the N-terminus of the mature protein would be the glutamine, Q, followed by the serine, and then the rest of the protein. This difference will be of no concern for those producing the fusion proteins by recombinant means, with the native amino acid leader sequence as is expected to be the case in most instances, as furin will cleave the natural leader sequence at the usual position, leaving the desired mature protein with the natural start of the ACE2 ectodomain. To assist the practitioner might wish to instead synthesize the fusion proteins, omitting the leader sequence, the sequence of fusion proteins using an exemplar linker and Fc region has been stated below starting from either the glutamine (SEQ ID NO:23 and SEQ ID NO:24) or from the serine as the N-terminus of the ACE2 ectodomain of the fusion protein (SEQ ID NO:21 and SEQ ID NO:22).

In some studies underlying the present invention, the leader sequence used in producing fusion proteins for the studies was: METDTLLLWVLLLWVPGSTG (SEQ ID NO: 13) (“Leader sequence 2”). In studies using this leader sequence, an aspartic acid (D) was used in place of the serine (S) present at the N-terminus of the wild-type ACE2 ectodomain (SEQ ID NO: 1). In vitro studies of fusion proteins made with the two leader sequences did not show any adverse impact on quality or activity of the fusion proteins. Further, the mature versions of MDR503-Fc (SEQ ID NO:10), MDR504-Fc (SEQ ID NO: 11), and MDR505-Fc (SEQ ID NO:12). fusion proteins started with aspartic acid in place of the serine in the native sequence, as shown in the respective sequences. Accordingly, fusion proteins starting with either aspartic acid (D) or serine (S) served as the N-terminus of the inventive fusion proteins. Given the results with fusion proteins containing aspartic acid, it is believed that glutamic acid (E) can be used in place of aspartic acid as the N-terminus of ACE2 ectodomain sequences for fusion proteins. Given the results with fusion proteins containing the non-polar amino acid serine, it is believed that the other non-polar amino acids without an aromatic ring, glutamine, threonine, and asparagine, can also be used as the N-terminus of the mutated ACE2 ectodomain sequences for fusion proteins.

Finally, it is noted that, as it is easiest to produce the inventive fusion proteins by recombinant expression, the proteins have been described with their leader sequences included. As practitioners will appreciate, the leader sequences are cleaved off by furin before or during secretion of the proteins and the mature proteins are then available to be purified and prepared for use as prophylactic or therapeutic agents. It is assumed that persons wishing to synthesize the inventive fusion proteins rather than produce them by recombinant expression can do so by simply omitting the leader sequences.

### The “A” (ACE2 Receptor Ectodomain) Component

The A component, the extracellular domain of the ACE2 receptor (as noted above, the extracellular domain is also referred to herein as the “ectodomain”) in the inventive fusion proteins is mutated from the wild-type ectodomain sequence (SEQ ID NO:1) in a manner that leaves almost all of the sequence intact, but that inactivates catalysis of angiotensin.

Three exemplar constructs were made in the course of the studies reported herein, in which one or both of two carefully chosen amino acid residues in the natural ACE2 ectodomain sequence (SEQ ID NO:1) were mutated to inactivate catalysis. The mutations made were: R273A, in the fusion protein usually referred to herein as MDR503-Fc (SEQ ID NO:10), H345A, in the fusion protein variously referred to herein as MDR504-Fc, MDR504 hACE2-Fc, the MDR504 construct, or MDR504 (SEQ ID NO:11), and R273AH345A, in the double mutant fusion protein variously referred to herein as MDR505-Fc, MDR505 hACE2-Fc, the MDR505 construct, or MDR505 (SEQ ID NO:12). In some of the embodiments of these inventive fusion proteins tested in the studies reported below, the sACE2 ectodomain started with an aspartic acid (D) at the N-terminus instead of the S normally present at that position, as shown in SEQ ID NO:1. Studies using like fusion proteins with the native S at the N-terminus showed no difference in quality or activity in the studies conducted; accordingly, it is believed that either amino acid residue can be used at the N-terminus. In some embodiments, the N-terminus of the mature fusion protein is a S. In some embodiments, the N-terminus of the mature fusion protein is a D. For clarity, it is noted that the positions of the residues identified above and in the inventive fusion proteins are with reference to the wild-type ACE2 ectodomain sequence (SEQ ID NO:1), starting with the M of the leader sequence, even though the leader sequence is cleaved before or during secretion of the mature protein. Persons of skill are accustomed to determining sequence position relative to an original sequence.

As noted, the substitutions made were of an alanine residue for an arginine at position 273, and of an alanine residue for the histidine normally present at position 345. Both arginine and histidine are positively charged at physiological pH, while alanine is neutral, non-polar, and has a hydrophobic side chain. While the exemplar amino acid, alanine, used in the studies has the shortest side chain of amino acids with those characteristics and is the most preferred for substitution of the arginine at position 273, the histidine at position 345, or of both residues, it is believed that other amino acids with those characteristics can also be used, with valine, isoleucine and leucine being preferred next to alanine, and amino acids having aromatic rings or a sulfur atom being less preferred. In some embodiments, glycine is used in place of the amino acid present at position 273 in the wild-type sequence (SEQ ID NO:1), or the one present at position 345 of the wild-type sequence (SEQ ID NO:1), or both.

### The “L” (Peptide Linker) Component

The L component is a short sequence of amino acids (3-20, more preferably 3-15, more preferably 3-12, more preferably 4-9, still more preferably 4-8 or 4-7, most preferably 4 amino acids in length ±1) that serves as bridge between the modified ACE2 receptor ectodomain and the Fc component.

In studies reported in the Examples, I-E-G-R (SEQ ID NO:2) was used as an exemplar linker. The present inventors have also made fusion proteins using the thrombin cleavage site, L-V-P-R-G-S (SEQ ID NO:4). More than one residue of the same amino acid can be used and, indeed, in some embodiments, all the residues in the linker can be the same, such as alanines (for example, A-A-A-A (SEQ ID NO:5) or A-A-A-A-A (SEQ ID NO:6)). The amino acids in the linker are preferably selected from those that are uncharged at physiological pH. If an amino acid that has a charge at physiological pH is used (as in the exemplar linker, I-E-G-R (SEQ ID NO:2), used in the studies reported below), there is preferably either just one (as in the thrombin cleavage site sequence (SEQ ID NO:4)) or, if two or more are used, there is preferably at least one residue with an opposite charge to keep the overall charge on the linker at 0 or ±1. For example, in the I-E-G-R (SEQ ID NO:2) linker, the acidic residue E is balanced by the basic amino acid R. If two or more charged amino acids are included in the linker, they are preferably separated by at least one amino acid. The amino acids selected for the linker also preferably do not contain aromatic side groups.

In a preferred embodiment, the linker is a flexible linker. In some of these embodiments, the flexible linker is a flexible “GS” linker, particularly those described by the formula (GGGGS)ₙ. Flexible GS linkers have been shown to improve the folding and stability of several fusion proteins. The use of peptide linkers in fusion proteins, and the use of GS peptides in particular, is reviewed in, e.g., Chen et al. (Adv Drug Deliv Rev. 2013 October 15; 65(10): 1357-1369. doi:10.1016/j.addr.2012.09.039) (“Chen 2013”). In a preferred embodiment, the GS linker is GGGGS (SEQ ID NO:15). In another preferred embodiment, the GS linker is GGGGSGGGGS (SEQ ID NO: 16). In another preferred embodiment, the GS linker is GGGGSGGGGSGGGGS (SEQ ID NO: 17). In another preferred embodiment, the GS linker is GGGGSGGGGSGGGGSGGGGS (SEQ ID NO:18).

Chen 2013 provides an extensive review of linkers that had been used in fusion proteins as of its 2013 publication date. It is expected that the practitioner is familiar with Chen 2013 and other literature concerning the use of linkers in fusion proteins, and is capable of selecting linkers ones suitable for use in the inventive fusion proteins. Any particular linker can be readily tested for its suitability with any particular combination of ACE2 ectodomain (SEQ ID NO: 1) and mutated Fc by substituting the test linker for the I-E-G-R (SEQ ID NO:2) linker used in some of the studies reported herein and seeing if the resulting fusion protein has similar pharmacokinetics, spike protein binding, and pseudovirus neutralization as those reported in the Examples. Linkers that have EC50s (ng/mL) approximately equal to or lower than that of the same fusion protein made with the same ACE2 ectodomain (SEQ ID NO:1) and mutated Fc made with the I-E-G-R (SEQ ID NO:2) linker and that do not otherwise cause adverse effects on the bioactivity or pharmacokinetics of the fusion protein, is satisfactory, while linkers that create fusion proteins with EC₅₀ s 20% or more higher than that of the same fusion protein made with the IEGR (She Q ID NO:2) linker or that otherwise causes adverse effects on the bioactivity or pharmacokinetics of the fusion protein is not satisfactory.

### The “F” (Fc) Component

The F component of the fusion proteins is a modified form the fragment crystallizable region (“Fc” or “Fc region”) of an antibody. In intact antibodies, the Fc region is the portion that interacts with cell surface receptors called “Fc receptors. The structure and sequences of immunoglobulin (“Ig”) variable and constant region domains and hinge regions for each of the Ig classes (IgG, IgM., IgA, IgE, and IgD) and of the two light chains, κ and λ, for each class, have been studied and elucidated for decades and are well known in the art. See, e.g., Goldsby et al., eds. KUBY IMMUNOLOGY, 4^{th} Ed., 2000, W. H. Freeman and Co., New York, Chapter 4. Some of the studies reported herein used constructs in which the Fc component of the fusion protein was IgG1 Fc.

Fusion proteins of the invention in which the Fc component is an IgG2 have also been made and tested in ELISA binding studies in the course of studies underlying the present disclosure. Constructs with an IgG2 Fc component are expected to show in vitro and in vivo results similar to those we observed for constructs with an IgG1 Fc component.

Preferably, the F component is the Fc region of a human immunoglobulin, which by definition may be considered to comprise the C_{H}2 and C_{H} 3 domains. The particular isoform of immunoglobulin (e.g., IgG or IgM) from which to select the C_{H}2 and C_{H}3 domains can be tailored depending on whether the practitioner wishes to administer the inventive fusion proteins (sometimes called “constructs” herein for ease of reference) to the deep aveolar space of the lungs or to the nasal passages and upper airway. For the deep aveolar space of the lungs, preferably the immunoglobulin is IgG and is of subclass 1 or 2. IgG is not only the predominant immunoglobulin found in the body, but is also the predominant immunoglobulin found in the alveolar space. For the nasal passages and upper airway, IgA could be a good choice. IgM is hard to produce by current methods, but can be used if desired. One advantage of IgM is that it is transported from the bloodstream into the gut by the polymeric immunoglobulin receptor (“pIgR”). Thus, it can be administered intravenously and the sACE2 component transported to the gut to treat an individual if the individual’s SARS-CoV-2 infection is manifesting in the intestinal tract.

Without wishing to be bound by theory, we believe that using the Fc region of an immunoglobulin normally found in the area to be protected by the inventive fusion proteins may result in favorable pharmacokinetics and reduce the possibility of side effects. It is anticipated that sequence of IgG1 heavy chain domains C_{H}2and C_{H}3 will be the sequence used for compositions to be used uniformly in the nasal passages, upper airway, and deep lung, as well as in intravenous applications.

The natural human antibody Fc sequence (the “wild type,” or “WT,” sequence) is preferred for embodiments in which the fusion proteins are intended for prophylactic use in patients who have not yet been infected with SARS-CoV-2, or who are in early stages of infection, as those embodiments are expected to cause Fc activation and result in clearing virus as quickly as possible. In patients who are presenting with chest x-ray showing lung involvement or with a cytokine storm, it is preferable to use embodiments of the fusion proteins in which the Fc receptor has been mutated (such as with the “LALA” mutations discussed below) to eliminate Fc activation. In some preferred embodiments, the sequence of the Fc domains is mutated from the WT sequence to eliminate FcRy binding, but to retain binding to the neonatal Fc receptor.

Antibody structure and binding have been studied extensively, as has engineering antibody heavy chains in general and constant regions in particular to address their binding to the five activating Fc receptors and the inhibitory Fc receptor to either enhance binding and complement activation or to ablate it. Mutations in the Fc region that enhance or that eliminate FcRy binding are reviewed in, e.g., Saunders, K., Front. Immunol., 2019, https://doi.org/10.3389/fimmu.2019.01296, which is incorporated herein by reference. See also, Schlothauer et al., Protein Eng Des Sel., 2016, (10):457-466.doi: 10.1093/protein/gzw040; Arduin et al., Mol Immunol, 2015; 63(2):456-63. doi: 10.1016/j.molimm.2014.09.017. Accordingly, it is expected that practitioners can select mutations in the Fc region that achieve the desired combinations of binding receptors in addition to the specific ones used in the studies reported in the Examples, below.

The mutations made in the Fc region are believed to improve both serum stability and transport of the fusion proteins from the circulation to the lung. The mutations are preferably of leucine (L) to alanine (A) substitutions at positions 234 and 235 of the immunoglobulin, a pair of mutations referred to as “LALA,” which eliminates FcyRI, IIa, and IIIa binding for both IgG1 and IgG4. See, e.g., Saunders, supra. An exemplar Fc of IgG1, containing the LALA mutation (SEQ ID NO:3), is shown in FIG. 10. As practitioners will appreciate, the residues mutated in the LALA mutations are numbered with respect to their positions in the native immunoglobulin, not the position they happen to occupy in the portion of the Fc region in the fusion protein In FIG. 10, the mutations appear at positions 18 and 19 of the portion of the immunoglobulin used in the fusion protein and set forth in the Figure. Other preferred mutations are LALA-PG (L234A L235A P329G, (SEQ ID NO:7)), and elimination of the glycosylation site at N297 (SEQ ID NO:8) (with or without a D265A substitution (SEQ ID NO:9)). Mutations in the Fc of subclasses of antibodies other than IgG1 that are known to eliminate FcRy binding can be used. In some embodiments, the wild type sequence can be used. See, e.g., Tao and Morrison, J. Immunol., 143:2595-2601 (1989); Chappel et al., Proc Natl Acad Sci USA 88:9036-9040 (1991); Lo et al., J Biol Chem. 292:3900-3908 (2017).

In embodiments intended for parenteral use, neonatal receptor binding is preferably retained, as it is believed to mediate longer serum half-life for the fusion proteins and the ability of the fusion proteins to leave the circulation and access organs such as the lung or kidney. In other embodiments to extend serum half-life, the Fc sequence can be mutated to forms known to extend half-life, such as the “YTE” mutations and others described by Booth et al., MABS, 2018, 10(7): 1098-1110, doi.org/10.1080/19420862.2018.1490119. Fusion proteins in which the YTE mutation of the Fc component has been made are a preferred embodiment for use in treating patients with either early or severe COVID-19.

The ability of the fusion proteins to bind the neonatal receptor is less important in fusion proteins intended for use in nasal sprays and aerosols, as in those embodiments, the fusion proteins are not being introduced into the circulation. In preferred embodiments, the mutations eliminate the ability of the Fc region to activate the complement-dependent cytotoxicity (CDC) pathway. See, Lo et al., supra. Without wishing to be bound by theory, in some embodiments, it may be desirable to retain the antibody-dependent, cell-mediated cytotoxicity (ADCC) pathway to activate natural killer cell activity in clearing virus.

The human genome has been sequenced and the sequences of the heavy chain constant region of each antibody class and subclass are set forth in GenBank. It is assumed that the practitioner is familiar with locating in GenBank and other resources the cDNA and the deduced amino acid sequence for a heavy chain constant region of an antibody of any selected class and subclass of interest.

### EXEMPLAR FUSION PROTEINS AND RESULTS

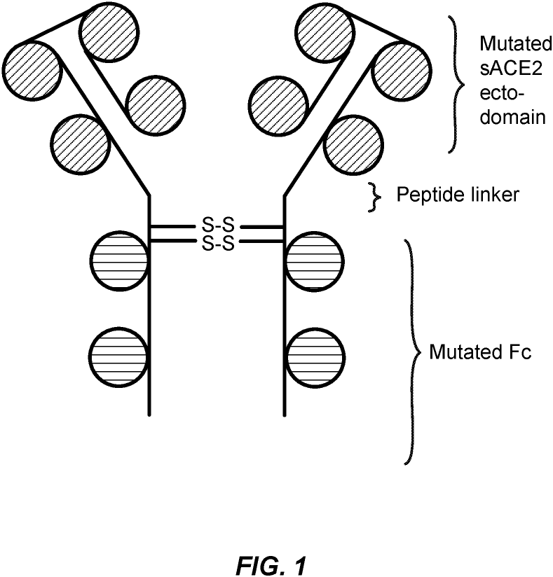

FIG. 1 presents a schematic representation of the inventive fusion proteins, including a mutated sACE2 ectodomain, a peptide linker, and a mutated Fc domain. FIG. 1 presents the fusion proteins as a homodimer. It is believed that the fusion proteins are expressed as monomers, but dimerize after secretion. Both monomers and dimers of the inventive fusion proteins are useful, as the mutated sACE2 ectodomains of each protein can bind a viral particle.

In the exemplar recombinant fusion proteins, the ACE2 ectodomain was engineered to inactivate the catalytic activation of angiotensin. To assist comparison of the effects of the individual mutations in the ACE2 ectodomain in the in vivo studies, the exemplar fusion proteins made for those studies all used the same linker, I-E-G-R (SEQ ID NO:2), and the same engineered Fc region of the same antibody isoform, or class, IgG, and within the class, the same subclass, IgG1. Fusion proteins for some in vitro studies of the ability of the fusion proteins to bind SARS-CoV-2 spike RBD protein, however, were made with a flexible “GS” linker, and some were made with an IgG2 Fc region. One exemplar fusion protein was made with an ACE2 ectodomain with the H345A mutation, a flexible GS linker, and an IgG1 Fc with the LALA mutations, while a second exemplar fusion protein was made with the same mutated ACE2 ectodomain and flexible GS linker, but with an IgG1 Fc with both the YTE mutation and the LALA mutations. Further, exemplar fusion proteins were made with the same mutated ACE2 ectodomain and flexible GS linker, but with an IgG2 Fc, one set with, and one set without, the YTE mutation.

While these are particularly preferred embodiments, other embodiments of the inventive fusion proteins can be made by using other linkers, such as those described in the section above on linkers, other subclasses of IgG, and, in some embodiments, classes of antibodies other than IgG.

When the human sACE2 R273A mutant with an aspartic acid in place of the serine at the N-terminus was expressed as a fusion protein linked through an exemplar peptide linker, I-E-G-R (SEQ ID NO:2), to the Fc region of IgG1 with LALA mutations (SEQ ID NO:3), we sometimes refer to the fusion protein as “MDR503 hACE2-Fc,” or as the “MDR503 construct.” When the human sACE2 H345A mutant with an aspartic acid in place of the serine at the N-terminus was expressed as a fusion protein linked through the same exemplar linker to the same mutated Fc region of IgG1 (SEQ ID NO:3), we sometimes refer to the fusion protein as “MDR504 hACE2-Fc,” as the “MDR504 construct,” or simply as “MDR504.” When the human sACE2 R273AH345A double mutant with an aspartic acid in place of the serine at the N-terminus was expressed as a fusion protein linked through the same exemplar linker to the same mutated Fc region of IgG1 (SEQ ID NO:3), we sometimes refer to the fusion protein as “MDR505 hACE2-Fc,” as the “MDR505 construct,” or simply as “MDR505.”

The fusion proteins were expressed and secreted using two exemplar mammalian cell lines, 293T cells and CHO cells. The nucleic acid sequences used for expression encoded the fusion proteins as proproteins, including a leader sequence which directs the preprotein to the secretory pathway, but which is cleaved by the proprotein convertase furin during the expression and secretion process and, accordingly, is not part of the mature protein secreted from the host cells. We did not sequence the mature fusion proteins used in the studies reported herein to determine the amino terminus of the mature protein, as the leader sequences were cleaved off naturally during the normal processes of expression and secretion. The results discussed below reporting the results of in vitro or in vivo studies of MDR504-Fc (SEQ ID NO:11) and MDR505-Fc (SEQ ID NO:12) are with respect to the mature proteins, without the leader sequences. Sequences of MDR504-Fc without the leader sequence (SEQ ID NO:19) and MDR505-Fc without the leader sequence (SEQ ID NO:20), and versions of MDR504-Fc without the leader sequence but commencing with a serine (“MDR504S-WL,” SEQ ID NO:21) or with a glutamine (“MDR504Q-WL,” SEQ ID NO:23), and versions of MDR505-Fc without the leader sequence but commencing with a serine (“MDR505S-WL,” SEQ ID NO:22) or with a glutamine (“MDR505Q-WL,” SEQ ID NO:24) are also set forth in the sequence listing. For clarity, given that “WL” could be read to designate amino acids with those single letter codes, it is noted that the “WL” in the names of the fusion proteins just mentioned stands for “Without Leader Sequence.”

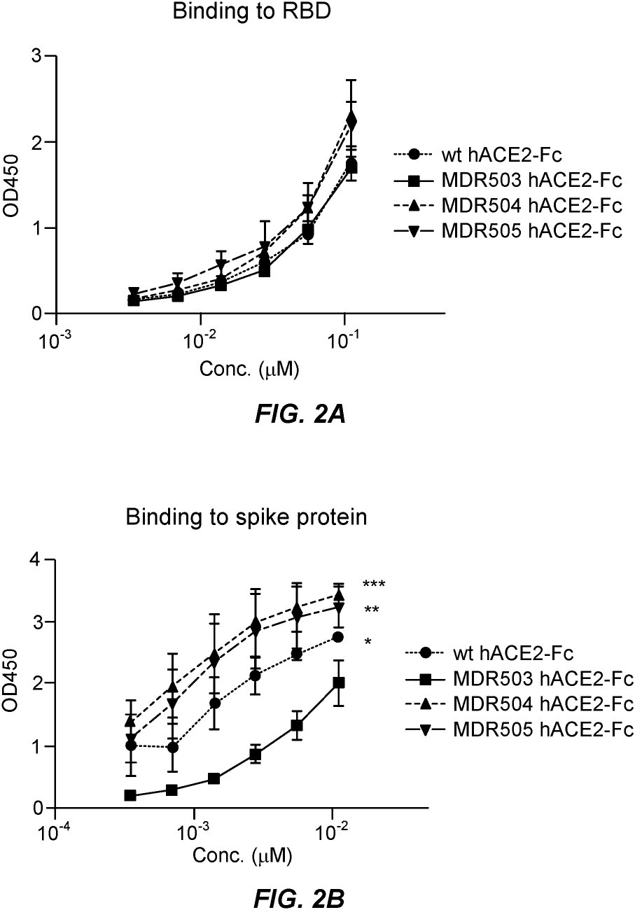

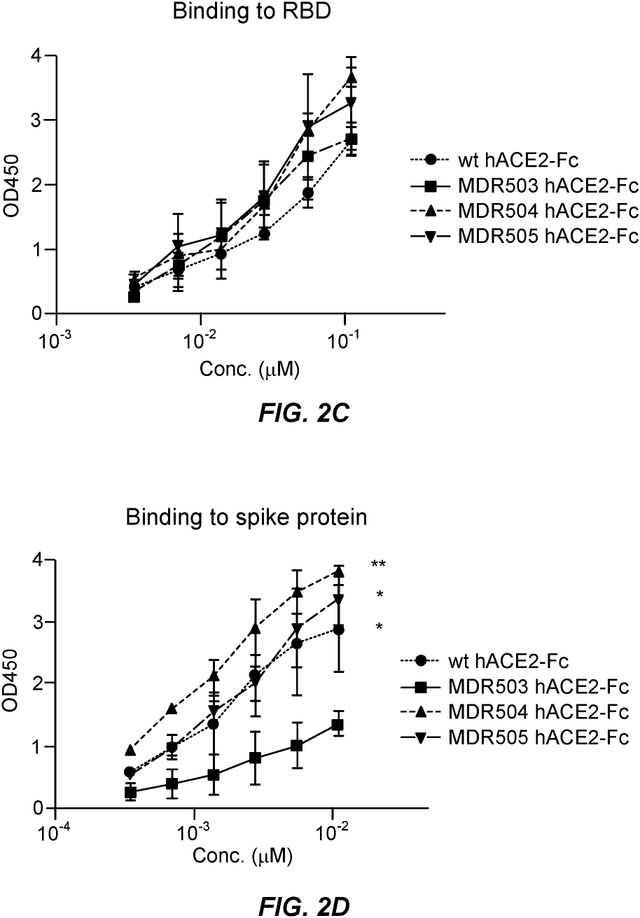

All the constructs were efficiently secreted after transient transfection in 293T cells and purified using protein G resin. All the fusion proteins bound monomeric SARS-CoV-2 receptor binding domain as well as trimeric spike protein. In experiments performed at room temperature, we observed higher binding of the MDR504 and MDR505 ACE2-Fc compared to WT ACE2-Fc (FIGS. 2A and 2B). This increase in binding was more dramatic when assayed at 37° C. (FIGS. 2C and 2D) where binding of the MDR504 mutant was superior against the trimeric spike protein.

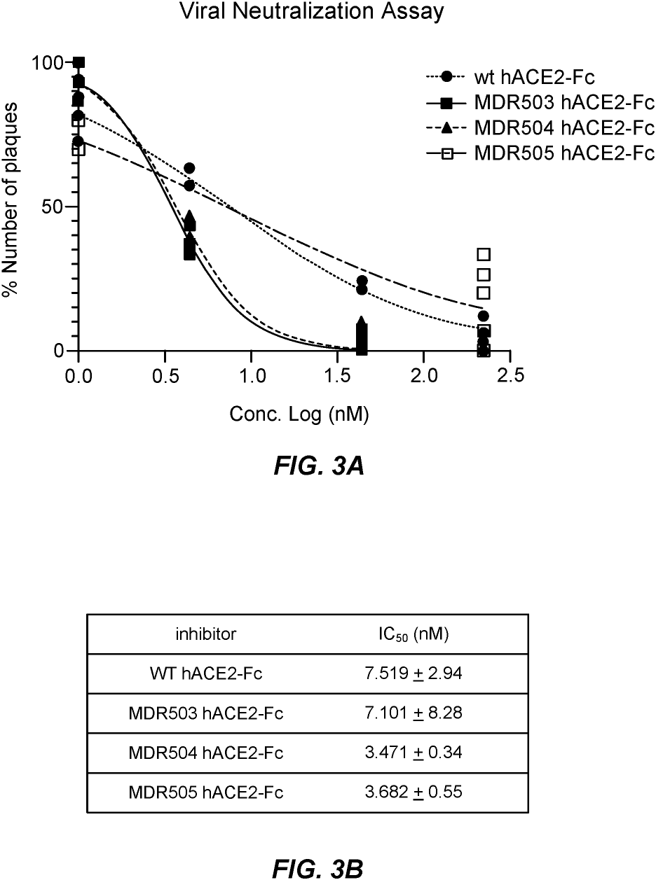

In in vitro studies using a pseudotyped model of SARS-CoV-2, we found superior neutralization with the MDR504 and MDR505 constructs compared to WT hACE2 Fc (FIGS. 8A and 8B). The dose response curve showed the excellent neutralization by MDR505 compared to WT hACE-Fc (WT hACE2-Fc; 8.58 nM vs MDR505hACE2-Fc; 2.01 nM) (FIGS. 8C and 8D). Next, we examined neutralization of SARS-CoV-2 using a plaque assay in Vero E6 cells. Initial studies were done at 50 µg/ml (223 nM) based on studies with Pavalizumab, an anti-RSV monoclonal antibody, showed that effective anti-RSV trough concentrations in vivo were ~ 40 µg/ml. Fifty µg/ml WT hACE2-Fc completely neutralized SARS-CoV-2 in vitro. Thus, we compared the wild-type protein to the MDR503, MDR504 and MDR505 constructs at lower concentrations. Here, MDR504 and MDR505 efficiently neutralized virus infection and had lower IC₅₀ s than the wild-type protein (FIGS. 3A and 3B). Without wishing to be bound by theory, we believe the MDR504 mutant, which has a single mutation, may be less immunogenic than the double mutant MDR505, and thus will be preferable for use in prophylactic applications in which fusion proteins may be administered over a longer period of time than those intended for therapeutic use. And, as shown in FIG. 7C and D, in further in vitro studies, MDR504 bound spike protein receptor binding domains (RBD) containing the sequences found in the UK and the South African SARS-CoV-2 variants at concentrations an order of magnitude or more lower than the concentrations needed by the same fusion protein, but using the wild-type ACE2 ectodomain.

While all three of the carefully designed recombinant fusion proteins we developed had advantageous properties, two, MDR504-Fc and MDR505-Fc had surprisingly good combinations of properties. It is believed that MDR505-Fc, in which the ACE2 ectodomain has both the mutation at position 345 of the MDR504 construct but also a mutation at position 273, will prove useful in preventing or treating infection with SARS-CoV-2 or with other coronaviruses that enter cells through binding ACE2.

MDR504-Fc stood out in the in vitro studies. This fusion protein had superior binding to the SARS-CoV-2 spike protein at both room temperature and at 37° C., and enhanced neutralization of virus in a Vero E6 cell plaque assay. While we believe the MDR505 construct will also be useful both as a prophylactic and as a therapeutic, constraints due in part to the pandemic itself kept us from being able to advance more than one construct into in vivo studies. Given the excellent results with MDR504-Fc in the in vitro studies, this was the construct advanced into in vivo studies.

In in vivo studies, MDR504-Fc continued to show surprisingly good results. As discussed further below, it had similar serum stability as wild-type sACE2-Fc, and was the highest among the constructs in levels that were secreted or transported into the fluid lining the epithelium of the lung after parenteral administration to C57B1/6 mice. This surprising and unique combination of features makes the mutated version of sACE2 used in the MDR504 construct a surprisingly advantageous mutated form of sACE2 to use in fusion proteins to prevent or to treat infection with SARS-Co-2.

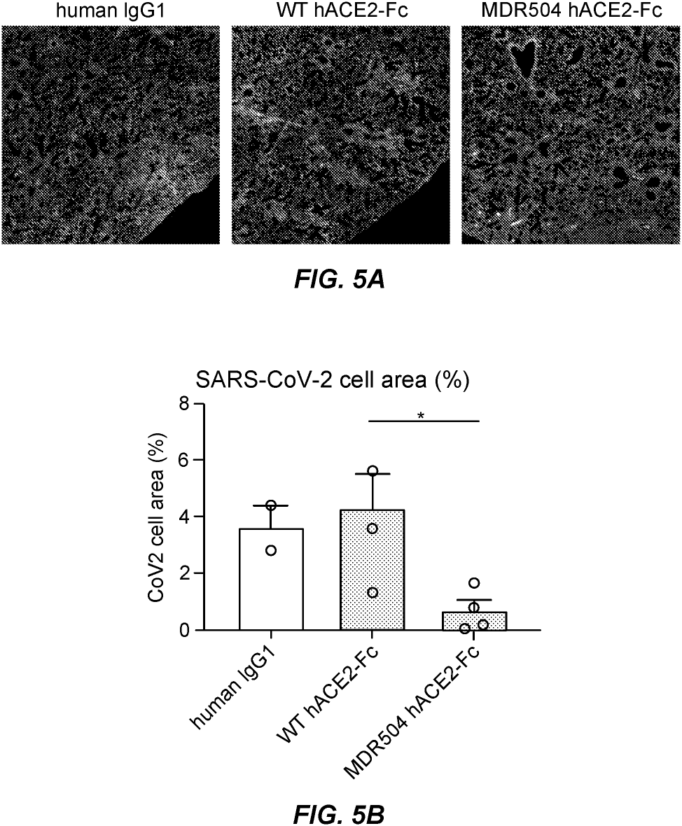

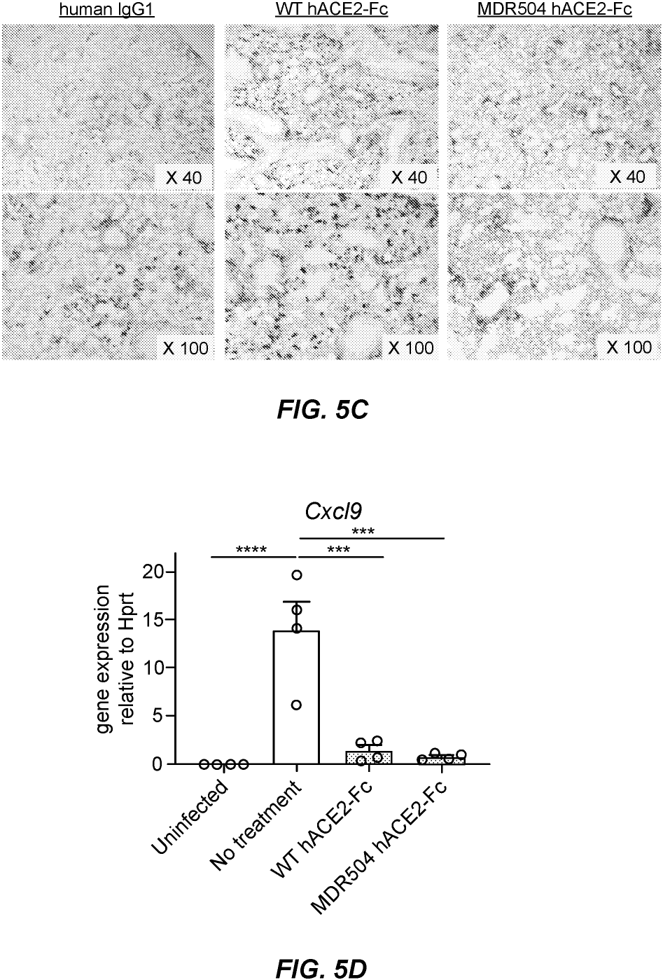

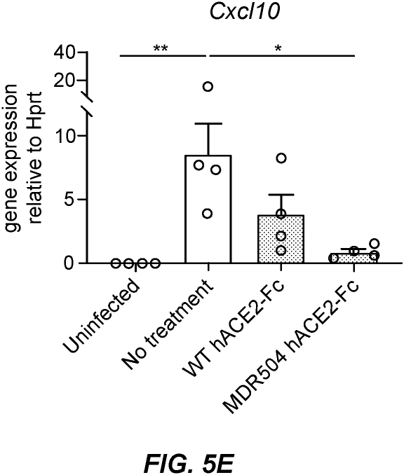

The good serum stability shown by the MDR504 construct when administered parenterally, and the transport of the MDR504 construct into the epithelial lining of the lung are both important properties for a potential agent to be used against a virus that infects both the lungs and other organs. Even more importantly, immunohistochemistry of tissues from mice treated with the MDR504 construct three to four hours before challenge with a SARS-CoV-2 infection showed significantly less infected cell area positive for SARS-CoV-2 following the challenge. (See, FIGS. 5A and 5B.) Infection by SARS-CoV-2 induces release of interferon stimulated cytokines such as Cxcl9 and Cxcl10. Mice treated with MDR504 hACE-Fc showed significantly reduced amounts of Cxcl9 and Cxcl10 gene expression compared to untreated mice and to mice treated with other constructs, including wt hACE2 (SEQ ID NO:1)-Fc. (See, FIGS. 5D and 5E.) Importantly, as shown in FIG. 6F, animals infected with SARS-CoV-2 and treated with MDR504 hACE-Fc had lower levels of VCAM-1 in pulmonary endothelial cells in blood vessels studied by immunohistochemistry, compared to the blood vessels of animals infected with SARS-CoV-2 and treated with a wild-type sACE2-Fc fusion protein or untreated. VCAM-1, a cell adhesion factor that mediates the adhesion of immune cells to vascular endothelium, is considered a surrogate marker for assessing endothelial dysfunction. Treatment with the exemplar MDR504-Fc construct significantly down-regulated the expression of VCAM-1 on pulmonary endothelial cells, indicating that the treatment protected the pulmonary endothelial cells from SARS-CoV-2 infection-mediated dysfunction and injury

The amino acid sequences of preproteins of some of the constructs studied in the Examples are set forth in FIGS. 10-12 (as discussed elsewhere, the preproteins contain leader sequences directing the preprotein to the secretory pathway and are naturally cleaved off by the host cell in the course of expressing and secreting the mature protein. FIG. 10 sets out the amino acid sequence of a fusion protein containing as components (a) the natural (“wild type”) hACE2 (SEQ ID NO:1) sequence of the ACE2 ectodomain, (b) the exemplar IEGR (SEQ ID NO.:2) linker, and the exemplar IgG1 Fc with LALA mutations (SEQ ID NO:3). Though shown separately in the Figure, the components are expressed as a single fusion protein, referred to herein variously as “wt hACE2-Fc,” “wt hACE2-Fc fusion protein,” “wt hACE2-Fc construct,” or “WT sACE2-Fc” (the presence of the linker between the ACE2 component and the Fc component is understood).

FIG. 11 sets out the amino acid sequence of a hACE2-Fc fusion protein in which the soluble hACE2 ectodomain sequence (SEQ ID NO:1) has been mutated to contain a mutation of R to A at the position corresponding to position 345 of the wild-type ACE2 sequence (including the leader, or signal, sequence), linked through the exemplar linker peptide IEGR (SEQ ID NO:2) (underlined in the Figure) to the exemplar IgG1 Fc with LALA mutations (SEQ ID NO:3). The resulting fusion protein is referred to herein as “MDR504-Fc” (SEQ ID NO:11), with the presence of the linker being understood. This fusion protein is also sometimes simply referred to herein as “MDR504 fusion protein,” “MDR504 construct,” or “MDR504.” As noted above, persons of skill will appreciate that the leader sequence of the fusion protein is cleaved off when the protein is recombinantly expressed, which is the expected method of production. The sequence of MDR504-Fc without the leader sequence is set forth as SEQ ID NO:19, and can be referred to as MDR504-WL-Fc, with the “WL” in these references meaning “without leader”. This is also the form of the fusion protein which would be made if the protein is made by synthesis rather than recombinant expression, and the form in which the protein would be administered whether made recombinantly or by synthesis. The sequence of MDR504-Fc without the leader sequence but commencing with a serine rather than an aspartic acid is SEQ ID NO:21, and can be referred to as “MDR504S-WL-Fc,” with the separation between the “S” of the serine and the “WL” helping clarify that, while the “S” does designate the amino acid serine, the “WL” is an acronym for “Without Leader” rather than the amino acid residues that can be represented by the same letters. Similarly, the sequence of MDR504-Fc, without the leader sequence but commencing with a glutamine rather than an aspartic acid is SEQ ID NO:23, and can be referred to as “MDR504Q-WL-Fc”.

FIG. 12 sets out the amino acid sequence of a sACE2-Fc fusion protein in which the natural soluble hACE2 ectodomain sequence (SEQ ID NO: 1) has been mutated to contain both (a) a mutation of arginine (R) to alanine (A) at the position corresponding to position 273 of the wild-type ACE2 sequence (including the leader, or signal, sequence), and (b) a mutation of histidine (H) to alanine (A) at the position corresponding to position 345 of the wild-type ACE2 sequence (including the leader, or signal, sequence), which ectodomain sequence is linked through an exemplar linker peptide, IEGR (SEQ ID NO:2), underlined. to the exemplar IgG1 Fc with LALA mutations (SEQ ID NO:3). The resulting fusion protein is referred to herein as “MDR505-Fc” (SEQ ID NO:12), with the presence of the linker being understood. This fusion protein is also sometimes simply referred to herein as “MDR505.” It is understood that the leader sequence of the fusion protein is cleaved off when the protein is recombinantly expressed, which is the expected method of production. The sequence of MDR505-Fc without the leader sequence is set forth as SEQ ID NO:20 and can be referred to as MDR505-WL-Fc, with the “WL” again standing for “Without Leader”, which is expected to be the form in which the fusion protein would be made if synthesized and the form in which the fusion protein will be administered. The sequence of MDR505-Fc without the leader sequence, and commencing with a serine rather than an aspartic acid, which can be referred as MDR505S-WL-Fc, is SEQ ID NO:22. The sequence of MDR505-Fc without the leader sequence but commencing with a glutamine rather than an aspartic acid is SEQ ID NO:24, and can be referred to as MDR505Q-WL-Fc.

### DEFINITIONS

As persons of skill will appreciate, the term “fusion protein” usually designates a protein produced by generating a synthetic nucleic acid sequence, cloning that sequence into an expression vector, introducing that expression vector into a suitable cell line, which may be bacterial or eukaryotic, having the transfected cells express the nucleic acid sequence, and purifying the resulting expressed protein. It is assumed that persons of skill are familiar with techniques standard in the art for performing each step in producing fusion proteins and can readily produce the inventive fusion proteins given the amino acid sequences and other guidance provided in this disclosure. Expression of the fusion proteins of this invention in eukaryotic cells is preferred, as they provide glycosylation of the proteins which cannot be provided by prokaryotic cells.

Amino acids and amino acid residues are referred to herein by their standard three letter or single letter abbreviations. Referring to the Fc region of human immunoglobulins, such regions have known sequences. Each amino acid at each position of the sequence can be assigned a number corresponding to its position in the sequence, when numbered starting from the amino terminus and proceeding to the carboxyl terminus of the peptide or protein. By convention, there is an agreed single letter code for each natural amino acid. By convention, the substitution of one amino acid in a sequence with another amino acid is described by stating in single letter code amino acid normally present at the position in the sequence at which the substitution is being made, stating the number of the position in the sequence, and then stating in single letter code the amino acid being substituted in for the original amino acid. Thus, the term “L234A,” with respect to a human IgG heavy chain, would be understood to refer to the change of the leucine (L) normally found at position 234 of the human IgG heavy chain sequence to an alanine (A).

The terms “effective amount” or “therapeutically effective amount” of a composition, as provided herein, refer to a nontoxic but sufficient amount of the composition to provide the desired therapeutic effect, or an amount sufficient to effect treatment of the subject, as defined below. The exact amount required will vary from subject to subject, depending on the species, age, and general condition of the subject, the severity of the condition being treated, and the particular macromolecule of interest, mode of administration, and the like. An appropriate “effective” amount in any individual case may be determined by one of ordinary skill in the art using routine experimentation.

The phrase “pharmaceutically acceptable,” in connection with administration of a substance to a human refers to a substance that is generally safe for human pharmaceutical use. In connection with administration to a non-human animal of a particular species, it refers to a substance that is generally safe and acceptable to a non-human animal of the species in question.

As used herein, the terms “pharmaceutically acceptable carrier” and “pharmaceutically acceptable vehicle” are interchangeable and refer to a fluid vehicle for containing OMP and enterotoxin compositions that can be injected into a host without adverse effects or administered to a host without adverse effects, depending on the intended route of administration. Suitable pharmaceutically acceptable carriers known in the art include, but are not limited to, sterile water, saline, glucose, dextrose, or buffered solutions. Carriers may include auxiliary agents including, but not limited to, diluents, stabilizers (i.e., sugars and amino acids), preservatives, wetting agents, emulsifying agents, pH buffering agents, viscosity enhancing additives, colors and the like.

### FORMULATIONS

Making inhalable dosage formulations to deliver agents to the nasal passages, to the lung, or to both, and dosages forms for administration of agents intravenously or subcutaneously are well known in the art. It is expected that persons of skill are familiar with the considerable literature and guidance that exists with respect to pharmaceutical formulations, as exemplified by texts such as Gennaro, A., REMINGTON’S PHARMACEUTICAL SCIENCES, 18^{th} Ed., (1990), Sheskey, Cook and Cable, eds. HANDBOOK OF PHARMACEUTICAL EXCIPIENTS, 8^{th} Ed. (Pharmaceutical Press, London, 2017), Perrie and Rades, eds., PHARMACEUTICS- DRUG DELIVERY AND TARGETING (Pharmaceutical Press, London, 2012), Chambers-Fox, REMINGTON EDUCATION: PHARMACEUTICS (Pharmaceutical Press, Philadelphia, 2014), and A. Adejare, ed., REMINGTON: THE SCIENCE AND PRACTICE OF PHARMACY, 23^{nd} Ed. (Academic Press, Philadelphia, 2020).

In addition to the fusion proteins, the formulation will typically include carrier of choice, which may be chosen depending on whether the formulation will be, for example, lyophilized or stored as a liquid for ready administration. The formulations may include one or more pharmaceutically acceptable excipients, stabilizers, binders, lubricants, fillers, buffers, antioxidants, preservatives, monosaccharides, disaccharides, and other sugars or carbohydrates, including glucose, mannose, sucrose, mannitol, trehalose or sorbitol.

In some embodiments, the inventive fusion proteins are used in the manufacture of a medicament for prophylactic administration to prevent a SARS-CoV-2 infection or to reduce the number of viral particles that infect the subject to whom the medicament is administered, thereby reducing the severity of any resulting infection. In some embodiments, the inventive fusion proteins are used in the manufacture of a medicament for therapeutic administration to a person with a SARS-CoV-2 infection, to reduce the number of viral particles available to spread within the subject to whom the medicament is administered, thereby reducing the severity or duration, or both of the infection. In some embodiments, the inventive fusion proteins are used in a product for prophylactic administration to prevent a SARS-CoV-2 infection or to reduce the number of viral particles that infect the subject to whom the medicament is administered, thereby reducing the severity of any resulting infection. In some embodiments, the inventive fusion proteins are used in a product for therapeutic administration to a person with a SARS-CoV-2 infection, to reduce the number of viral particles available to spread within the subject to whom the medicament is administered, thereby reducing the severity or duration, or both of the infection.

### ROUTE, DOSING, AND ADMINISTRATION

For purposes of the inventive methods, an “effective amount” of an administration of an embodiment of the inventive fusion proteins refers to an amount of the fusion proteins that, alone or in combination with further doses, produces the desired response. With respect to use as a prophylactic agent, e.g., in capturing SARS-CoV-2 viruses, an effective amount is an amount that prevents infection upon exposure to SARS-CoV-2 viruses or that reduces the severity of the response the subject would otherwise have had upon exposure to a given number of SARS-CoV-2 virus particles.

For use in ameliorating or otherwise treating a subject with a SARS-CoV-2 infection, the “therapeutically effective amount” will generally be the same as that used for prophylactic use. “Ameliorating or otherwise treating” a subject with a SARS-CoV-2 infection means reducing the severity or progression of a SARS-CoV-2 infection (that is, COVID-19) compared to like individuals who had a similar severity of an infection with COVID-19, but who were not administered an embodiment of the fusion proteins.

In some embodiments, administration of the fusion proteins is by spraying, or inhaling, a spray or an aerosol into the nasal cavity, nasopharynx, or both. In some embodiments, administration of the fusion proteins is by inhalation of an aerosol or spray through the mouth into the lungs, for example, from an inhaler. In some embodiments, administration of the fusion proteins to a patient is through an endoscope into one or both of the patient’s lungs.

Administration by each of these routes is well known in the art. For example, Flumist® is a commercially available flu vaccine administered by spray into the nose. Each dose is provided in a sprayer. According to the prescribing information, “the tip attached to the sprayer is equipped with a nozzle that produces a fine mist that is primarily deposited in the nose and nasopharynx.” If desired, the mist or aerosol can be delivered to the posterior nose and paranasal sinuses by pulsating airflow. See, e.g., Moller, et al., Aerosol Med Pulm Drug Deliv, 2014, 27(4):255-63.doi: 10.1089/jamp.2013.107. In other embodiments, the fusion proteins can be administered over time by intranasal drug delivery devices. Such devices are discussed in, for example, Dkhar et al., Pharm Dev Technol, 2018 23(3):282-294.doi: 10.1080/10837450.2017.1389956. Epub 2017 Nov 2.

With regard to inhaling powders and sprays into the lungs, inhalers have been available for decades that allow asthma patients to self-administer therapeutic agents. The available inhaler types include pressurized metered dose inhalers (“MDIs”), breath-activated inhalers, inhalers with spacer devices, and nebulisers, which turn a solution containing the fusion proteins into a fine mist. It is contemplated that each of these types of inhalers can be readily adapted to deliver unit dose formulations of the inventive fusion proteins to the lungs of persons in need thereof.

Endoscopic introduction of agents into the bronchii or other portions of the lung has typically been performed to provide cancer chemotherapy by local injections into tumors by microneedles and the like. See, e.g., Steinfort, D., Respirology, 2018; 23(4):352-353.doi: 10.1111/resp.13222. Epub 2017 Nov 17. Administration of aerosols or mists through endoscopes has not necessarily been needed to date, as inhalation though inhalers has been so successful in having drugs distributed throughout the lungs, but interventional endoscopic techniques would allow local administration of the inventive fusion proteins to the lungs if a practitioner felt that this was necessary for a particular patient given their clinical situation.

Administration of the fusion proteins to the nasal passages, by inhalation of aerosols into the lung, or by endoscopic introduction results in a high local concentration of the fusion proteins at the site of action. Accordingly, the dosing is expected to be modest: 0.5-3 mg/kg. Thus, for a child, a typical dosage will be 50-180 mg, for a teenager or small adult 0.1 gm to 0.36 grams (gm), for an average-sized adult, 0.15 gm to 0.54 mg, and for a large or obese adult, 0.20 gm to 0.72 gm.

In embodiments in which the patient is showing signs of damage to the kidneys or other organs that are affected by covid-19 infections, the fusion proteins may be administered instead by intravenous infusion. It is expected that the dosage of fusion proteins for i.v. infusion will be 5-30 mg/kg. Thus, for a child, a typical dosage will be 500 mg-1.80 g, for a teenager or small adult 1 gm to 3.6 g, for an average-sized adult, 1.5 g to 5.4 g, and for a large or obese adult, 2 gm to 7.2 gm. In some embodiments, the fusion proteins are provided at 5- 30 mg/kg body weight in an intravenous solution administered over 15-30 minutes.

### EXAMPLES

### Example 1

This Example sets forth methods and materials used in the studies discussed below.

### Mice

Male wild type C57BL/6J mice or female K18-hACE2 humanized mice 6-10-weeks-old were used for in vivo studies. The mice were bred in-house or purchased from The Jacks on Laboratory. All experiments were performed using sex- and age-matched controls and approved by the Institutional Animal Care and Use Committee of Tulane University.

### Generation of Different Constructs of Human ACE2 Fusion Proteins

The DNA sequences of the extracellular domains of ACE2 and IgG1 were synthesized by Genscript and cloned into pcDNA3.1. Transient transfection was performed using Lipofectamine™ 3000 Transfection Reagent (Invitrogen, Thermo Fisher Scientific Inc., Waltham, MA) in 293T cells. The collected supernatants were collected and purified by protein G-sepharose (Thermo Fisher, Thermo Fisher Scientific Inc., Waltham, MA). The concentration and purity were confirmed by measuring the UV absorbance at wavelength of 280 nm, BCA assay (Thermo Fisher) and human IgG ELISA (Thermo Fisher).

### Western Blotting

In some studies, following the removal of the supernatants, cell lysates were dissolved in PBS (50 mg/ml) containing protease inhibitors (Thermo Scientific, Thermo Fisher Scientific Inc., Waltham, MA) and 1 mM PMSF. In some studies, following the removal of the supernatants, cell lysates were dissolved in 50 mg/ml cell lysis buffer (Cell Signaling Technology, Inc., Danvers, MA) containing protease inhibitors (Thermo Scientific) and 1 mM PMSF. BCA assay was performed to quantify protein and 5.0 µg protein was used for Western blotting. Western blots were performed using 7.5% SDS-PAGE gels (Bio-Rad, Hercules, CA) under non-reducing or reducing conditions with 2.5% 2-mercaptoethanol and transferred to PVDF membranes. The blot was probed with goat anti-human IgG-HRP (Southern Biotech, Birmingham, AL). After incubation with IgG-HRP-conjugated anti-human antibody, membranes were washed and incubated with SuperSignal West Pico Chemiluminescent Substrate (Thermo Scientific). Signal was detected using a Bio-Rad ChemiDoc MP imaging system.

### ELISA for Human ACE2 and Spike Protein

ELISA plates were coated with 2 µg/mL spike glycoprotein receptor binding domain (RBD) from ATCC (BEI RESOURCES) or recombinant S1 subunit RBD (RayBiotech Life, Peachtree Corners, GA) overnight at 4° C. Coated plates were washed with washing buffer (0.05% Tween 20 in PBS), blocked for 2 h at room temperature with blocking buffer (1% BSA and 0.1% Tween 20 in PBS), and washed before the addition of the supernatants or cell lysates from transfected 293T cells. After 2 h incubation at room temperature (“RT”), or 1 h incubation at 37° C., the plates were washed and incubated with goat anti-human IgG conjugated with horseradish peroxidase (Southern Biotech) diluted 1/5,000 in assay diluent (0.5% BSA and 0.05% Tween 20 in PBS) for 1 h at RT, or for 30 min at 37° C., TMB peroxidase substrate (Southern Biotech) was added to each well. Absorbance was read at 450 nm on a microplate reader (BioTek U.S., Winooski, VT).

### Pseudovirus Production

Pseudoviruses for antibody screening were generated using the following plasmids: The spike protein of SARS-CoV-2 (pcDNA3.1(+)-SARS2-S), and the HIV-1 pro-viral vector pNL4-3.Luc.R-E- were obtained through the NIH AIDS Research and Reference Reagent Program. The pseudoviruses were produced by transient co-transfection of 293T cells using a polyethyleneimine (PEI)-based transfection protocol. Five hours after transfection, cells were washed with phosphate-buffered saline (PBS), and 20 mL of fresh media was added to each 150 mm plate. Twenty-four hours post transfection, the supernatant was collected and filtered through a 0.45 µM pore size filter and stored at 4° C. prior to use.

### Pseudovirus Neutralization Assay

Targeted 293T cells were transfected with pcDNA3.1(+)-humanACE2 and pCSDest-TMPRSS2 plasmids for 6 h. The cells were then trypsinized and seeded 1×10⁵ cells/well in DMEM complete into 96-well plates (100 µL/well), then incubated for 16 hours at 37° C. and 5% CO₂. SARS-1, SARS-2, and VSV pseudovirions were incubated with the test samples at room temperature for 1 h, and then added to the target cells in 96-well plates. Plates were incubated for 48 hr at 37° C. and 5% CO₂ and levels of viral infection were determined by luminescence using the neolite reporter gene assay system (PerkinElmer, Waltham, MA). Virus alone was used as a control and data was normalized to the control.

### Human ACE2 Neutralization of SARS-CoV2 by Plaque Assay

Vero E6 cells were plated in a 6 well plate at 8 × 10⁵ cells per well and incubated overnight. Each construct of hACE2-Fc was preincubated with SARS-CoV2 virus for 10 minutes before infection in 1 ml media. The cells were washed once with PBS and infected at a MOI of 0.01 with the compounds for 1 h. Following infection, the supernatants containing the virus and compounds were removed and 3 ml overlay media containing each compound were added to the wells and incubated for an additional 4 days. Post infection, the cells were fixed and stained to visualize plaques.

### Pharmacokinetics Study

C57BL/6 mice were injected with 4 mg/kg body weight WT ACE2-Fc or the MDR504 mutant intravenously via retro-orbital and serum was collected at 0, 1, 24, or 72 h. Mice were euthanized at 6 h and 72 h and underwent bronchoalveolar lavage (BAL) to measure ACE2-Fc in the epithelial lining fluid of the lung. The concentration of hACE2-Fc was analyzed by detecting human IgG-Fc by ELISA. Purified anti-human IgG Fc antibody (Biolegend, Inc., San Diego, CA) was used as a capture antibody and anti-Human IgG Fc, Multi-Species SP ads-HRP (SouthernBiotech)was used as a detection antibody, other procedures were as described above.

### In Vivo Prophylaxis and Treatment Studies of MDR504

All animals were cared for in accordance with the NIH guide to Laboratory Animal Care. The Institutional Biosafety Committee approved the procedures of sample handling, inactivation, and removal from a BSL3 containment. A murine model of SARS-CoV2 was used. C57BL/6 mice were first oropharyngeally inoculated with 1.5 × 10⁹ pfu adenovirus encoding human ACE2 (Vector Biosystems Inc., Malvern, PA) as described in Han et al., Am J Respir Cell Mol Biol, 2021, 64(1):79-88. Four days later, mice received 2 × 10⁵ pfu (prophylaxis model) or 2 × 10³ pfu (treatment model) of SARS-CoV-2 intranasally and were euthanized 72 hours post infection by ABSL3 staff. To evaluate MDR504, mice were dosed intravenously via retro-orbital with 15 mg/kg body weight polyclonal human IgG1, WT ACE2-Fc or MDR504 4 hours prior to SARS-CoV-2 infection (prophylaxis study) or 18 hours post SARS-CoV-2 infection (treatment study). As a treatment study of high viral load, we also confirmed efficacy using K18-hACE2 humanized mice, which we infected with 2 × 10⁵ pfu SARS-CoV-2 and treated with 30 mg/kg body weight of fusion proteins 4 hours post infection.

### In Vivo Evaluation of MDR504 by Immunohistochemistry

Lung tissues were collected in Zinc formalin (Anatech Ltd., Battle Creek, MI), embedded in paraffin, and 5 µm thick sections were cut, adhered to charged glass slides, baked overnight at 56° C. and passed through Xylene, graded ethanol, and double distilled water to remove paraffin and rehydrate the tissue sections. A microwave was used for heat induced epitope retrieval. Slides were heated in a high pH solution H-3301 antigen unmasking solution (Vector Laboratories, Inc., Burlingame, CA), rinsed in hot water and transferred to H-3300, a heated low pH solution (Vector Laboratories), where they were allowed to cool to room temperature. Sections were washed in a solution of phosphate-buffered saline and fish gelatin (PBS-FSG) and transferred to a humidified chamber. Tissue was blocked with 10 % normal goat serum (NGS) for 40 minutes, followed by a 60 minute incubation with polyclonal guinea pig anti-SARS-CoV-2 antibodies (1:1000) (NR-10361, BEI Resources, Manassas, VA). Slides were transferred to the humidified chamber and incubated, for 40 minutes, with secondary antibodies tagged with various Fluor fluorochromes and diluted to a working concentration of 2 µg/mL. Slides were mounted using a homemade anti-quenching mounting media containing Mowiol (#475904, MilliporeSigma, Burlington, MA) and DABCO (#D2522, SigmaAldrich, St. Louis, MO) and imaged with a Zeiss Axio Slide Scanner (Carl Zeiss Microscopy, LLC, White Plains, NY). Whole slide images of the lungs were analyzed with computer software (HALO, Indica Labs, Albuquerque, NM) using a tissue segmentation algorithm, trained with a deep convolution neural network (VGG), by a board certified veterinary pathologist to recognize cellular inflammation within the lungs of mice.

### Real-Time RT-PCR

RNA was isolated with RNeasy Plus Mini Kit (QIAGEN) post phase separation using Trizol reagent (ThermoFisher), and cDNA was prepared using iScript reverse transcriptase master mix (Bio-Rad). Real-time RT-PCR was carried out with a Bio-Rad CFX96 system using TaqMan® PCR Master Mix (ThermoFisher Scientific) and Cxcl9 or Cxcl10 premixed primers/probe sets from Thermo Fisher Scientific. A FAM labeled probe targeting the N1 amplicon of N gene (2019-nCoV RUO Kit, Cat. No. 10006713, IDT-DNA, Coralville, IA) of SARS-CoV-2 (accession MN908947) was utilized following the manufacturer’s instructions. Subgenomic mRNA (sgmRNA) encoding the N gene was quantified following the assay published by Wolfel et al., Nature, 2020, 581:465-469. The viral copy numbers from the lung samples are represented as copies/100ng of RNA.

### RNA Scope

Z-fix-fixed and paraffin-embedded lung sections underwent in situ hybridization according to the manufacturer’s instructions (Advanced Cell Diagnostics, Inc., Newark, CA). Briefly, after hydrogen peroxide treatment, we performed target retrieval, created a hydrophobic barrier, and applied AP conjugated-nCoV2019-S probe (RNAscope® Probe V-nCoV2019-S, ACD cat# 848561) and Cxcl9 probe (RNAscope® Probe Mm-Cxcl9-C2, ACD cat# 489341-C2) for hybridization. After hybridization, tissues were stained with 50% Hematoxylin.

### Statistical Analysis

Statistical analysis was performed with GraphPad Prism 8.0 (GraphPad Software, San Diego, CA). P values < 0.05 was evaluated statistically significant. Comparisons between two normally distributed groups were performed by simple 2-tailed unpaired student’s t-test. For multiple groups comparisons, we used one-way or two-way ANOVA with Tukey’s post-hoc analysis. Values are represented as means ± SEM. P values are annotated as follows (*) ≤0.05, (**) ≤0.01, (***) ≤0.001, and (****) ≤0.0001.

### Example 2

This Example sets forth results of studies underlying the present disclosure.

### Expression of hACE2-Fc Constructs

All constructs of hACE2-Fc expressed proteins consistent with homodimers after transfection in HEK293T cells. After transient transfection of the constructs in 293T cells, human IgG-Fc in the supernatant was readily detected by ELISA. Proteins were run on a reduced SDS-PAGE gel and migrated at ~ 140 kDa consistent with the predicted molecular weight of the monomer. Also, SDS-PAGE analysis in non-reducing conditions revealed migration consistent with a dimeric protein.

### SARS-CoV2 RBD and Spike Binding

All constructs were efficiently secreted after transient transfection in 293T or CHO cells and purified using protein G resin. All proteins bound monomeric SARS-CoV-2 receptor binding domain as well as trimeric spike protein.

In experiments run at room temperature, we observed higher binding of the MDR504 and MDR505 hACE2-Fc and a lesser extent to the MDR503 hACE2-Fc compared to wild-type ACE2-Fc (FIGS. 2A and 2B). This increase in binding was more dramatic when assayed at 37° C. (FIGS. 2C and 2D) where binding of the MDR504 mutant was superior against the trimeric spike protein.

### Neutralization of SARS-CoV2 Infection

Using a pseudotyped model of SARS-CoV-2 we found superior neutralization with the MDR504 mutant and the MDR505 double mutant compared to wild type human ACE2 Fc IgG1, as shown in FIGS. 8A and 8B. The dose response curve showed excellent neutralization by MDR505 (WT hACE2; 8.58 nM vs MDR505 hACE2; 2.01 nM), as shown in FIGS. 8C and 8D.

Next, we examined neutralization of SARS-CoV-2 using a plaque assay in Vero E6 cells. Initial studies were done at 50 µg/ml (223 nM) based on studies with Pavalizumab, an anti-RSV monoclonal antibody, showed that effective anti-RSV trough concentrations in vivo were ~ 40 µg/ml (Beeler and van Wyke Coelingh, J Virol, 1989, 63: 2941-2950). Fifty µg/ml of WT hACE2-Fc completely neutralized SARS-CoV-2 in vitro. Then, we compared the wild-type sACE2 construct to the MDR5033, MDR504, and MDR505 constructs. MDR504 and MDR505 efficiently neutralized virus infection and had lower IC50s than the wild-type protein (FIGS. 3A and 3B). cleavage site.

### Example 3

This Example sets forth the results of in vivo studies of the MDR504 fusion protein.

### Serum Stability and BAL Fluid Concentrations

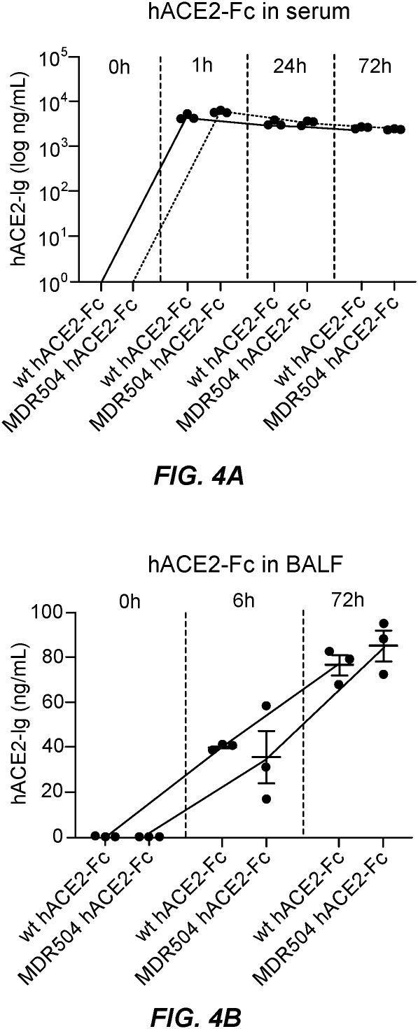

After a single IV injection, the MDR504 mutant had similar serum stability as WT ACE2-Fc (FIG. 4A), but we detected higher levels in the epithelial lining fluid of the lung after parenteral administration to C57BL/6 mice (FIG. 4B). The MDR504 mutant had a slightly higher peak concentration in serum and a half-life of approximately 145 h.

### Testing of the MDR504 Construct as a Prophylactic Treatment

We next tested the efficacy of engineered hACE2-Fc as a prophylactic treatment in our recently established mouse model (Sun et al., Cell, 2020, 182:734-743 e735; Han et al., Am J Respir Cell Mol Biol, 2021, 64(1):79-88). Mice were untreated or dosed intravenously with 15 mg/kg with human polyclonal IgG1, WT hACE2-Fc, or MDR504 four hours prior to SARS-CoV-2 infection. Histological analysis of untreated mice, human IgG1 treated mice, and WT hACE2-Fc treated mice showed similar widespread SARS-CoV-2 infection in the distal lung, with approximately 4% of the cell area infected (naive lung (%); 4.32 ± 0.37, human IgG1 treated lung (%); 3.59 ± 0.80, WT hACE2-Fc treated lung (%); 4.29 ± 0.80). In contrast, mice treated with MDR504 had significantly less infected cell area positive for SARS-CoV-2 staining (MDR504 hACE2-Fc treated lung (%); 0.64 ± 0.37) (FIGS. 5A and 5B). RNA scope was performed for viral RNA and showed significant fewer infected cells in mice treated with MDR504 (FIG. 5C). Infection with SARS-CoV-2 induces interferon stimulated chemokines such as Cxcl9 and Cxcl10 similar to infected human lung (Liao et al., Nat. Med. 2020, 26:842-844). Notably, in MDR504 treated mice, the Cxcl9 gene expression was relatively reduced by RNA Scope (FIG. 5C) and the Cxcl9 and Cxcl10 gene expression was substantially down-regulated as shown by RT-PCR (FIGS. 5D, 5E).

### Testing of the MDR504 Construct as a Therapeutic Intervention

We next examined MDR504 in treatment models in which we administered MDR504 fusion proteins to animals after challenge with SARS-CoV-2. Based on more potent binding of MDR504 fusion proteins produced in CHO cells, as shown by ELISA (FIG. 9A) and better pharmacokinetics (FIGS. 9B and 9C), we used CHO cells to produce the MDR504 fusion proteins tested in the treatment studies. We confirmed that MDR504 fusion protein produced in CHO had a molecular weight nearly identical to that of the MDR504 fusion protein produced in 293T cells (FIG. 9D). As noted above, it is believed that any differences in molecular weight are due to differences in glycosylation patterns due to expressing the fusion protein in two different cell types.

To evaluate MDR504 as a treatment, we tested it in a SARS-CoV-2 high-dose (2 × 10⁵ pfu) infection model in K18-hACE2 humanized mice. As K18-hACE2 mice support significantly higher viral replication than the Ad5hACE2 model, we administered MDR504 or control 4 hours after viral infection and increased the dose to 30 mg/kg body weight. We have documented that sub-genomic RNA is readily detectable at four hours, demonstrating that lung epithelial cells are already infected at this time point. In the high-dose SARS-CoV2 model, MDR504 substantially suppressed viral load, as measured by total and subgenomic N gene expression (FIGS. 6A and 6B) and Cxcl9 gene expression (FIG. 6C). Moreover, administration of MDR504 significantly reduced weight loss (FIG. 6D) and improved survival (FIG. 6E) in the mice compared to mice given either no treatment or treatment with the WT hACE2-Fc fusion protein.

### Example 4

This Example reports the results of testing the MDR504 for its ability to bind the spike protein found in some variants of SARS-CoV-2.

### Binding of MDR504 to SARS-CoV-2 Variants

Lastly, we tested MDR504 binding to several spike variants that have been associated with reduced binding by casirivimab, imdevimab, or bamlanivimab. MDR504 retained pM binding to these variants (FIG. 7A) with only mildly reduced binding to V483A (FIG. 7B). We observed enhanced binding of MDR504 to ACE2 to variants Y543F as well as HV69-70del / N501Y / D614G that were identified in SARS-CoV-2 variants. We also observed increased binding to the UK variant (B.1.1.7) that was significantly shifted to the left compared to wild-type ACE2 (FIGS. 7C and 7D). Binding to the South African variant was also significantly higher with MDR504 versus wild-type ACE2.

### Example 5

This Example discusses the results set forth in the preceding Examples.

One potential mechanism that has made SARS-CoV-2 more infectious than the 2002 SARS-CoV epidemic is the affinity of the SARS-CoV-2 spike protein for the human ACE2, which has been hypothesized to be higher than that of SARS-Co-V (Hoffman, et al., Cell, 2020, 181:271-280 e278; Shang et al. Nature, 2020. 581(7807):221-224. doi: 10.1038/s41586-020-2179-y). However, this potentially increased affinity also represents a potential therapeutic target for blocking viral entry.

We made ACE2-Fc fusions with mutations in the ACE2 catalytic domain, as well as in the IgG1 constant region to abrogate FcRy binding. Interestingly, we found that the MDR504 mutation had greater binding affinity for the SARS-CoV-2 RBD and spike protein. This translated to a lower IC₅₀ value for infectious viral neutralization and in a pseudovirus assay.

Cryo-EM studies of SARS-CoV-2 RBD has been shown to bind the NH2 terminus of human ACE2. Lan et al., Nature, 2020, 581:215-220. However, the RBD also binds to residues K353, G354, and D355 (Lan et al., supra), and thus it is possible that the MDR504 mutation in the catalytic domain affects this binding. Thus, the MDR504 mutation may have a structural basis for enhanced neutralization over wild-type ACE2. Importantly our constructs showed activity against infectious SARS-CoV-2. The MDR504 mutant also showed excellent stability in serum and achieved therapeutic levels in bronchoalveolar lavage (BAL) fluid in a murine PK/PD study. This exemplar fusion protein also demonstrated strong results when administered to prevent infection by SARS-CoV-2 and as a therapeutic intervention when administered after exposure to a high dose of the virus.

Taken together, these results show that the inventive fusion proteins are useful for both pre- and post-exposure prophylaxis and as a therapy for persons with COVID-19, whether caused by the original viral strain or some or all of the variants that have emerged as of this writing. The inventive fusion proteins are particularly likely to be useful for therapeutic use in early stages of SARS-CoV-2 infection.

The inventive fusion proteins are expected to be useful both as a complement to vaccines, and as stand-alone prophylaxis or therapy for subjects that may not be good candidates for vaccines, such as patients with hematologic or other malignancies, those undergoing immunosuppressive therapy for organ transplantation, those with autoimmune disease, and those with HIV or other conditions that prevent them from mounting a robust immune response to a vaccine.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A composition comprising a fusion protein of formula 1, 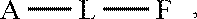
wherein
“A” is a modified extracellular domain of a human ACE2 receptor, which extracellular domain has a wild-type sequence having an N-terminal amino acid residue and an ability to catalyze angiotensin, which extracellular domain has been mutated to reduce or to eliminate said ability of said extracellular domain to catalyze angiotensin,
“L” is a short peptide linker, and
“F” is the fragment crystallizable region (“Fc” or “Fc region”) of an antibody, optionally wherein said Fc region has been mutated to eliminate FcRγ binding, but to retain binding to a neonatal Fc receptor.

2. The composition of claim 1, wherein said N-terminal amino acid residue is a serine, glutamine, aspartic acid, glutamic acid, threonine, or asparagine residue.

3. (canceled)

4. The composition of claim 1, wherein said mutated human ACE2 extracellular domain has a non-polar, uncharged amino acid residue at a position corresponding to position 345 of SEQ ID NO:1.

5. The composition of claim 4, wherein said non-polar, uncharged amino acid residue at said position corresponding to position 345 of SEQ ID NO:1 is valine, isoleucine, leucine, glycine, or alanine.

6. (canceled)

7. The composition of claim 1, wherein said mutated human ACE2 extracellular domain has a non-polar, uncharged amino acid residue at a position corresponding to position 273 of SEQ ID NO:1.

8. The composition of claim 7, wherein said non-polar, uncharged amino acid residue at said position corresponding to position 273 of SEQ ID NO:1 is valine, isoleucine, leucine, glycine, or alanine.

9. (canceled)

10. The composition of claim 1, wherein said mutated human ACE2 extracellular domain has a non-polar, uncharged amino acid residue at both said position corresponding to position 273 of SEQ ID NO:1 and at said position corresponding to position 345 of SEQ ID NO:1, which non-polar, uncharged amino acid residue at each position is the same or different.

11. The composition of claim 10, wherein said non-polar, uncharged amino acid residue at said position corresponding to position 273 of SEQ ID NO:1 and at said position corresponding to position 345 of SEQ ID NO:1 is an alanine.

12. The composition of claim 1, wherein said short peptide linker is from 3 to 20 amino acids in length.

13-14. (canceled)

15. The composition of claim 1, wherein said short peptide linker is I-E-G-R (SEQ ID NO:2), L-V-P-R-G-S (SEQ ID NO:4), A-A-A-A (SEQ ID NO:5), A-A-A-A-A (SEQ ID NO:6), has the formula GGGGS(n) or has the formula GGGGS (SEQ ID NO:15)._{.}

16-17. (canceled)

18. The composition of claim 1, wherein said Fc is of IgG1, mutated to have alanines in place of leucines at positions 234 and 235 (SEQ ID NO:3).

19. The composition of claim 1, wherein said fusion protein is MDR504-Fc (SEQ ID NO:11), MDR504-WL-Fc (SEQ ID NO:19), MDR504S-WL-Fc (SEQ ID NO:21) or MDR504Q-WL-Fc (SEQ ID NO:23), MDR505-Fc (SEQ ID NO:12), MDR505-WL-Fc (SEQ ID NO:20), MDR5O5S-WL-Fc (SEQ ID NO:22) or MDR505Q-WL-Fc (SEQ ID NO:24 ).

20-22. (canceled)

23. The composition of claim 1, wherein said Fc region has been mutated to contain LALA mutations, a YTE mutation, or both LALA mutations and a YTE mutation.

24. The composition of claim 1, further comprising a pharmaceutically acceptable carrier, a pharmaceutically acceptable excipient, or both.

25-40. (canceled)

41. A product comprising a composition of claim 1 for the prevention of, or the reduction of severity of, an infection by a coronavirus that binds to human ACE2 receptor.

42. The product of claim 41, wherein said coronavirus is SARS-CoV-2.

43. The product of claim 41, wherein, wherein said composition of claim 1 is MDR504-Fc (SEQ ID NO:11), MDR504-WL-Fc (SEQ ID NO:19), MDR504S-WL-Fc (SEQ ID NO:21), or MDR504Q-WL-Fc (SEQ ID NO:23), MDR505-Fc (SEQ ID NO:12), MDR505-WL-Fc (SEQ ID NO:20), MDR5O5S-WL-Fc (SEQ ID NO:22), or MDR505Q-WL-Fc (SEQ ID NO:24).

44-46. (canceled)

47. The product of claim 41, wherein said Fc of said composition of claim 1 has LALA mutations, a YTE mutation, or both LALA mutations and a YTE mutation.

48. The product of claim 41, wherein said composition is in a pharmaceutically acceptable carrier, is mixed with a pharmaceutically acceptable excipient, or both.

49. A method of preventing or of ameliorating an infection in a subject by a coronavirus that binds to a human ACE2 receptor, said method comprising administering to said subject an effective amount of a pharmaceutical composition comprising a fusion protein of Formula 1, 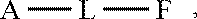
wherein “A” is a mutated extracellular domain of a human ACE2 receptor, which extracellular domain has a wild-type sequence has having an N-terminal amino acid residue and an ability to catalyze angiotensin, which wild-type sequence of said extracellular domain has been mutated to reduce or to eliminate said ability of said extracellular domain to catalyze angiotensin,
“L” is a short peptide linker, and
“F” is the fragment crystallizable region (“Fc” or “Fc region”) of an antibody, optionally wherein said Fc region has been mutated to eliminate FcRγ binding, but to retain binding to a neonatal Fc receptor.

50. The method of claim 49, wherein said N-terminal amino acid residue is a serine, glutamine, aspartic acid, glutamic acid, threonine, or asparagine residue.

51. (canceled)

52. The method of claim 49 wherein said mutated human ACE2 extracellular domain has a non-polar, uncharged amino acid residue at a position corresponding to position 345 of SEQ ID NO:1.

53. The method of claim 49, wherein said non-polar, uncharged amino acid residue at said position corresponding to position 345 of SEQ ID NO:1 is valine, isoleucine, leucine, glycine, or alanine.

54. (canceled)

55. The method of claim 49, wherein said mutated human ACE2 extracellular domain has a non-polar, uncharged amino acid residue at a position corresponding to position 273 of SEQ ID NO:1.

56. The method of claim 49, wherein said non-polar, uncharged amino acid residue at said position corresponding to position 273 of SEQ ID NO:1 is valine, isoleucine, leucine, glycine, or alanine.

57. (canceled)

58. The method of claim 49, wherein said mutated human ACE2 extracellular domain has a non-polar, uncharged amino acid residue at both said position corresponding to position 273 of SEQ ID NO:1 and at said position corresponding to position 345 of SEQ ID NO:1, which non-polar, uncharged amino acid residue at each position is the same or different.

59. The method of claim 49, wherein said non-polar, uncharged amino acid residue at said position corresponding to position 273 of SEQ ID NO:1 and at said position corresponding to position 345 of SEQ ID NO:1 is an alanine.

60. The method of claim 49, wherein said short peptide linker is from 3 to 20 amino acids in length.

61-62. (canceled)

63. The method of claim 49, wherein said short peptide linker is I-E-G-R (SEQ ID NO:2), L-V-P-R-G-S (SEQ ID NO:4), A-A-A-A (SEQ ID NO:5), A-A-A-A-A (SEQ ID NO:6), or has the formula GGGGS(n), or G-G-G-G-S (SEQ ID NO:15)._{.}

64-65. (canceled)

66. The method of claim 49, wherein said Fc is of IgG1, mutated to have alanines in place of leucines at positions 234 and 235 (SEQ ID NO:3).

67. The method of claim 49, wherein said fusion protein is MDR504-Fc (SEQ ID NO:11), MDR504-WL-Fc (SEQ ID NO:19), MDR504S-WL-Fc (SEQ ID NO:21) or MDR504Q-WL-Fc (SEQ ID NO:23) MDR505-Fc (SEQ ID NO:12), MDR505-WL-Fc (SEQ ID NO:20), MDR505S-WL-Fc (SEQ ID NO:22) or MDR505Q-WL-Fc (SEQ ID NO:24).

68-70. (canceled)

71. The method of claim 49, wherein said Fc of said composition of Formula 1 has LALA mutations, a YTE mutation, or both LALA mutations and a YTE mutation.

72. The method of claim 49, further wherein said composition comprises a pharmaceutically acceptable carrier.


SEQUENCE LISTING (24 sequences)

SEQ ID NO: 1 - protein, 740 aa, human
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 2 - protein, 4 aa, Artificial
  IEGR peptide linker
IEGR

SEQ ID NO: 3 - protein, 231 aa, Artificial
  Human Fc region with LALA-PG mutations
PKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 6 aa, Artificial peptide
  Exemplar linker peptide 2
LVPRGS

SEQ ID NO: 5 - protein, 4 aa, Artificial
  Examplar linker peptide 3
AAAA

SEQ ID NO: 6 - protein, 5 aa, Artificial
  Exemplary peptide linker 4
AAAAA

SEQ ID NO: 7 - protein, 231 aa, Artificial; identical to SEQ ID NO: 3
  Homo sapiens Fc region with LALA-PG mutations

SEQ ID NO: 8 - protein, 230 aa, Artificial
  Human Fc region with N297 deletion
PKSCDKTHTCPPCPAPELLGGPSVFLFPPKPKDTLMISRTPEVTCVVVDVSHEDPEVKFNWYVDGVEVHNAKTKPREEQYSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 231 aa, Artificial
  Human Fc region with N297 del and D265A mutations
PKSCDKTHTCPPCPAPELLGGPSVFLFPPKPKDTLMISRTPEVTCVVVADVSHEDPEVKFNWYVDGVEVHNAKTKPREEQYSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 978 aa, Artificial
  Fusion protein with ACE2 extracellular domain with leader sequence and R273A mutation, IEGR linker, and Fc region with LALA mutations
METDTLLLWVLLLWVPGSTGDTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGAFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSIEGRPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 977 aa, Artificial
  Fusion protein comprising (1) ACE2 extracellular domain (showing leader sequence cleaved off before use), (2) IEGR linker, and (3) Fc region with LALA mutations
METDTLLLWVLLLWVPGSTGDTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCAPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSIEGRPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 977 aa, Artificial
  Fusion protein comprising (1) ACE2 extracellular domain, showing leader sequence cleaved off before use, (2) IEGR peptide linker, and (3) Fc region with LALA mutations.
METDTLLLWVLLLWVPGSTGDTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGAFWTNLYSLTVPFGQKPNIDVTDAMVDQAWDAQRIFKEAEKFFVSVGLPNMTQGFWENSMLTDPGNVQKAVCAPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSIEGRPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 18 aa, Homo sapiens
MSSSSWLLLSLVAVTAAQ

SEQ ID NO: 14 - protein, 20 aa, Artificial
  Leader sequence used in MDR503, MDR504, and MDR505 constructs
METDTLLLWVLLLWVPGSTG

SEQ ID NO: 15 - protein, 5 aa, Artificial
  Flexible GS linker
GGGGS

SEQ ID NO: 16 - protein, 10 aa, Artificial
  Flexible GS peptide linker, 2 repeats
GGGGSGGGGS

SEQ ID NO: 17 - protein, 15 aa, Artificial
  Flexible GS linker, 3 repeats
GGGGSGGGGSGGGGS

SEQ ID NO: 18 - protein, 20 aa, Artificial
  Flexible GS peptide linker, 4 repeats
GGGGSGGGGSGGGGSGGGGS

SEQ ID NO: 19 - protein, 957 aa, Artificial
  MDR504-Fc, without leader sequence
DTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCAPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSIEGRPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 957 aa, Artificial
  MDR505-Fc, without leader sequence
DTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGAFWTNLYSLTVPFGQKPNIDVTDAMVDQAWDAQRIFKEAEKFFVSVGLPNMTQGFWENSMLTDPGNVQKAVCAPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSIEGRPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 957 aa, Artificial
  MDR504S-WL-Fc
STIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCAPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSIEGRPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 957 aa, Artificial
  MDR505S-WL-Fc
STIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGAFWTNLYSLTVPFGQKPNIDVTDAMVDQAWDAQRIFKEAEKFFVSVGLPNMTQGFWENSMLTDPGNVQKAVCAPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSIEGRPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 958 aa, Artificial
  MDR504Q-WL-Fc
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCAPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSIEGRPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 958 aa, Artificial
  MDR505Q-WL-Fc
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGAFWTNLYSLTVPFGQKPNIDVTDAMVDQAWDAQRIFKEAEKFFVSVGLPNMTQGFWENSMLTDPGNVQKAVCAPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSIEGRPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK